(19) United States
(12) Patent Application Publication
(10) Pub. No.: US 2023/0011889 A1
(43) Pub. Date: Jan. 12, 2023

(54) GENETICALLY ENGINEERED DOUBLE NEGATIVE T CELLS AS AN ADOPTIVE CELLULAR THERAPY

(71) Applicant: University Health Network, Toronto (CA)

(72) Inventors: Li Zhang, Toronto (CA); Jong Bok Lee, Toronto (CA); Daniel Vasic, Vaughan (CA); Ismat Khatri, Toronto (CA); Dalam Ly, Toronto (CA); Yuki Sze Long Leung, Markham (CA)

(21) Appl. No.: 17/782,071

(22) PCT Filed: Dec. 7, 2020
(86) PCT No.: PCT/CA2020/051682

§ 371 Date: Jun. 2, 2022

Related U.S. Application Data
(60) Provisional application No. 62/944,634, filed on Dec. 6, 2019.

(51) Int. Cl. A61K 35/17; C07K 14/725; C12N 5/0783; A61P 35/00

(52) U.S. Cl. CPC A61K 35/17; C07K 14/7051; C12N 5/0636; A61P 35/00; C07K 2319/03; C12N 2510/00

(57) ABSTRACT

The disclosure relates to the development and use of CD4− CD8− double negative T (DNT) cells genetically modified to bind to one or more target antigens to enhance DNT cell anti-cancer activity such as with a chimeric antigen receptor (CAR). Genetically modified DNT cells can be generated ex vivo and expanded from allogeneic healthy donor cells and used as off-the-shelf therapy to overcome allogeneic graft-versus-host disease (GvHD) and/or host-versus-graft rejection in the treatment of cancer.

### RELATED APPLICATIONS

This application claims priority to U.S. provisional patent application No. 62/944,634 filed Dec. 6, 2019, the entire contents of which are hereby incorporated by reference.

### FIELD

The present disclosure relates to cellular therapy and more specifically double negative T-cells that have been genetically modified to bind to one or more target antigen(s) for use in adoptive cellular therapy.

### INTRODUCTION

Allogeneic hematopoetic stem cell transplantation (allo-HSCT) is a potential curative treatment for patients with high-risk hematopoetic malignancies and is associated with higher disease-free survival rate than the conventional chemotherapy (Alatrash and Molldrem, 2009). Donor-derived T cell-mediated anti-leukemic effect contributes to the increased survival in patients as T cell-depleted grafts results in higher relapse rate (Alatrash and Molldrem 2010). However, the use of allo-HSCT in the clinic is limited by a shortage of suitable donors, the toxicity of the treatment, and other associated complications, particularly graft-versus-host disease (GvHD) (Shlomchik 2007, Alatrash and Molldrem 2010). GvHD is largely caused by donor-derived lymphocytes that recognize normal tissues of the patients as foreign (Shlomchik 2007). Subsequently, potent immune responses can be induced toward normal tissues, resulting in tissue damage and, possibly, in the death of the patients in severe cases (Shlomchik 2007, Alatrash and Molldrem 2010). Consequently, GvHD is a major obstacle that limits the use of cellular therapies in an allogenic setting.

Nevertheless, clinical benefits achieved with allo-HSCT have provided the foundation for scientists to explore the anti-leukemic functions of immune cells. Cancer immunotherapy is now considered the fourth pillar of cancer treatment along with chemotherapy, radiotherapy, and surgery. In particular, the effectiveness of adoptive cellular therapy (ACT) using T cells to treat different hematological and solid malignancies has been demonstrated in multiple clinical studies (Tran, Robbins et al. 2016, Park, Riviere et al. 2018). Advances in technologies such as genetically modifying immune cells to express a chimeric antigen receptor (CAR) or a transgenic T cell receptor and use of artificial antigen presenting cells have been implemented to improve the therapeutic potency of ACTs (Maus, Thomas et al. 2002, Kalos and June 2013, Harris and Kranz 2016). In particular, genetic modification of autologous patient T cells using CD19-CAR technology led to tremendous successes in treating B cell leukemia and lymphoma patients. Complete remission (CR) rates of up to 90% have been achieved in refractory or relapsing CD19+ B cell acute lymphoblastic leukemia (B-ALL) patients, where the expected response rate is approximately 30% with the conventional chemotherapy (Neelapu, Locke et al. 2017, Schuster, Svoboda et al. 2017, Maude, Laetsch et al. 2018). CD19-CAR T cell therapy has been FDA approved for clinical use for these diseases (Ruella and Kenderian 2017, 2018). However, with increasing numbers of patients treated with CAR19-T cell therapies, limitations of the current forms have become apparent. Sophisticated expansion methods cause uncertainty of producing therapeutically relevant numbers of T cells; time required for cell expansions allow disease progression that may lead to patient death before treatment; and requirement of clinically-approved facilities and highly trained personnel for cell expansion have led to inconsistency of manufactured cellular products and high production costs (June, Riddell et al. 2015, Dwarshuis, Parratt et al. 2017, Ren, Liu et al. 2017). Collectively, the current forms of CAR-T cell therapy are highly costly and inaccessible to many patients, making them practically challenging for a wide clinical translation. Also, downregulation of CD19 is a common mechanism of resistance employed by B-ALL to escape CAR19-mediated cytotoxicity, contributing to a relatively short median survival of 12.9 months despite the high initial response rate (Park, Riviere et al. 2018).

Double negative T cells (DN T cells or DNTs) are mature peripheral T lymphocytes that express the CD3-TCR complex but do not express CD4, CD8, or NKT cell markers; they represent 1˜3% of peripheral blood mononuclear cells (PBMC) (Zhang, Yang et al. 2000).

Protocols for expanding DNTs from healthy donors (HD) have been described and DNTs have significant anti-leukemic activity against various cancer types (Lee, Minden et al. 2017). DNTs induced killing of the allogeneic acute myeloid leukemia (AML) blasts in a dose-dependent manner through a perforin/granzyme-dependent pathway (Merims, Li et al. 2011, Lee, Minden et al. 2017). The use of allogeneic immune cells is known to have the risk of unwanted allogeneic immune responses, such as GvHD and host-versus-graft (HvG) rejection. However, in animal models, it has been shown that unlike conventional CD4+ or CD8+ T cells, infusion of allogeneic mouse DNTs does not cause GvHD (Zhang, Yang et al. 2000, Young, DuTemple et al. 2003, He, Ma et al. 2007). Consistent with these findings, in patients receiving allo-HSCT, diminished incidence and severity of GvHD were correlated with higher frequencies of DNT cell levels in the blood (McIver, Serio et al. 2008). More recently, it was demonstrated that allogeneic DNTs expanded from HD peripheral blood (PB) are not toxic towards normal cells, and infusion of DNTs into mice does not cause xenogeneic GvHD (Lee, Minden et al. 2017). Further, allogeneic DNTs co-persist with conventional T cells without developing alloreactivity in vitro and in a xenograft model, suggesting that DNTs can avoid HvG rejection (Lee, Kang et al. 2019). Further, DNTs targeted an array of hematological cancer targets in a donor-independent manner, collectively supporting the use of DNTs as an off-the-shelf cellular therapy.

The current forms of CAR-T products are produced in a highly personalized manner, broad applicability of CAR-T therapies are restricted by high production costs, batch-to-batch inconsistencies and complicated logistics, which have driven the need to generate new CAR-T therapies that can be used in an off-the-shelf manner (Dwarshius, Parrat et al. 2017, Torikai, Cooper et al. 2016). Recently, the number of studies exploring the use of ACT as an off-the-shelf treatment has grown (Torikai, Cooper et al. 2016, Ruella, Kenderian et al. 2017, Poirot, Ren et al. 2015, Ren et al. 2017, Qasim et al. 2017, Boyiadzis et al. 2017, Sheridan, C. et al. 2018, Suck et al. 2016, Zeng, Tang et al. 2017). As an off-the-shelf ACT is not patient-specific, cellular products can be pre-manufactured as a readily available treatment. Also, validation of cellular products manufactured at a larger scale in centralized facilities increases product consistency and reliability at a lower cost (Dwarshius, Parrat et al. 2017, Torikai, Cooper et al. 2016, Ruella, Kenderian et al. 2017). However, an effective clinically-applicable off-the-shelf allogenic T cell therapy should meet the following criteria: 1) expandable to a therapeutic number under clinically-compliant conditions; 2) can target an array of cancers in a donor-unrestricted manner; 3) does not cause graft vs. host disease (GvHD); 4) is able to avoid HvG rejection; and 5) storable under current Good Manufacturing Practice (cGMP) conditions without hampering its function (June, Riddell et al. 2015). Several different methods have been in pre-clinical development to meet these criteria, including the use of different cellular products (Boyiadzis et al. 2017, Suck et al. 2016, Zeng, Tang et al. 2017, Li, Hermanson et al. 2018, Deniger et al. 2014); however, issues with scalability, persistence, or feasibility of repeated infusions of allogeneic immune cell products into patients remain to be resolved (Boyiadzis et al. 2017). Currently, the most mature off-the-shelf CAR-transduced cellular products, termed Universal CAR19 T (UCART19) cell involve the use of genetic tools to disrupt the αβTCR receptor on conventional T cells (T_{conv}) (Poirot, Ren et al. 2015, Ren et al. 2017, Qasim et al. 2017), the main mediator of GvHD. Additionally, CD52 is disrupted through gene editing to make the UCART19 cells resistant to a strong immunosuppression drug Alemtuzumab, an antibody drug that broadly depletes CD52-expressing immune cells. In order to enable persistence of allogeneic UCART19, patients are treated with the alemtuzumab for prolonged immunosupression, which however increases risks of various immunosuppression adverse events such as infections. Though a heavily invested strategy that is currently in clinical trials (Qasim et al. 2017, Sheridan, C. 2018), incomplete modification has led to detrimental GvHD in ALL patients treated with UCART19 cells (Qasim et al. 2017). Other approaches propose ideas such as knocking out HLA class I to avoid HvG rejection, however, these cells may be prone to NK-cell mediated rejection due to ‘missing-self’ signal. Further, introducing additional genetic modifications will lower the product yield and increase the cost of CAR-T manufacturing, which already imposes a significant barrier to the wide application of CAR-T therapies. Collectively, this highlights the potential issues and added complexity that can arise from relying on additional genetic modifications to develop off-the-shelf CAR-T therapy.

### SUMMARY

In one aspect, there is provided a double negative T (DNT) cell that has been genetically modified to bind to one or more target antigen(s) as well as associated uses of the genetically modified DNT cells for the treatment of cancer. The target antigens may be any suitable antigens, for example an antigen enriched or preferentially expressed on the surface of a cancer cell. In one embodiment, there is provided a double negative T (DNT) cell that has been modified to express a nucleic acid molecule encoding a chimeric antigen receptor (CAR) that binds to the target antigen. In one embodiment, the DNT cell is CD4−, CD8−, CD3+, γδ-TCR+ and/or αβ-TcR+.

In one embodiment, the target antigen is an antigen expressed on a cancer cell. In one embodiment, the CAR comprises an extracellular antigen-binding domain that binds to a target antigen expressed on a cancer cell. For example, in one embodiment, the target antigen is selected from CD4, CD33, CD19, CD20, CD123, LeY, Mesothelin, EGFR, ROR1, EpCam, MUC1, HER1/2, MET/HGF, neoantigens (driver, non-driver), MAGE family, and NY-ESO-1. In one embodiment, the target antigen is CD4. In one embodiment, the target antigen is CD19. Optionally, the DNT cells described herein may be genetically modified to bind to a plurality of target antigens such as by modifying the cells to express a plurality of CARs that target different antigens. Also provided are methods and uses of the CAR-DNT cells described herein for the treatment of cancer.

As shown in the Examples, DNT cells modified to express a CAR against CD19 (CAR19) retained comparable expansion profiles as those expanded without CAR transduction. Furthermore, in vivo experiments using CAR19-DNTs in a mouse xenograft model showed a dose-dependent effect in reducing leukemia load and prolonged survival. Notably, CAR19-DNTs were also shown to exhibit a cytotoxic effect in the absence of CD19 expression on target cells, confirming their dual cytotoxic function and the ability of CAR-DNTs to target cancers that may exhibit reduced expression of target antigens following or during treatment.

In addition, while both non-transduced allogenic DNTs and CD19 CAR-transduced DNTs have been demonstrated not to cause graft-versus-host disease (GvHD), it was unknown whether CAR modification of DNTs will result in a host-versus-graft reaction and/or alloreactivity. Remarkably, while conventional CAR T_{conv} cells primed with allo-antigens elicited strong killing of allogeneic donor cells, CAR-DNTs did not develop cytotoxicity against allogeneic cells (FIGS. 7a and 7b). Also, allogeneic PBMC co-cultured with CAR-T_{conv} cells developed alloreactivity as significantly higher numbers of CAR-T_{conv} cells were killed by PBMCs, whereas those co-cultured with CAR-DNTs did not develop cytotoxicity toward CAR-DNTs upon subsequent co-culture.

Accordingly, in one embodiment there is provided a method of treating cancer in a subject in need thereof, the method comprising administering to the subject an effective amount of a population of double negative T (DNT) cells that have been genetically modified to bind to a target antigen. Also provided is the use of an effective amount of a population of DNT cells that have been genetically modified to bind to a target antigen for treating cancer in a subject in need thereof. Also provided is a method of treating CD4+ cancers in a subject in need thereof, the method comprising administering to the subject an effective amount of a population of DNT cells that have been genetically modified to bind to a CD4 target antigen. Also provided is the use of an effective amount of a population of DNT cells that have been genetically modified to bind to a CD4 target antigen for treating CD4+ cancer in a subject in need thereof. Also provided are DNT cells or a population of DNT cells that have been genetically modified to bind to a target antigen as described herein as well as pharmaceutical compositions comprising the genetically modified DNT cells. In one embodiment, the DNT cells are genetically modified to express a nucleic acid molecule encoding a chimeric antigen receptor (CAR) that binds to the target antigen.

In one embodiment, the population of DNT cells comprises or consists of allogenic cells, optionally from one or more healthy donors. In one embodiment, the DNT cells used for producing the genetically modified DNT cells are pooled DNT cells from a plurality of donors. In one embodiment, the population of genetically modified DNT cells does not induce graft-versus-host disease (GvHD) in the subject. In one embodiment, the population of genetically modified DNT cells induces less GvHD in the subject relative to conventional CD4+ CD8+ T cells (T_{conv} cells). In one embodiment, the population of genetically modified DNT cells avoids or suppresses host-versus-graft (HvG) rejection in the subject. In one embodiment, the population of genetically modified DNT cells suppresses HvG rejection in the subject relative to CAR-T_{conv} cells. In one embodiment, the population of genetically modified DNT cells persists in the subject for longer than 2 weeks, 3 weeks, or 4 weeks. In one embodiment, the population of genetically modified DNT cells persists in the subject for longer than a control population of CD4+ CD8+ CAR T cells, optionally for longer than 2 weeks, 3 weeks, or 4 weeks. In one embodiment, the subject does not receive immunosuppressive therapy following administration of the population of genetically modified DNT cells. For example, in one embodiment the subject does not receive immunosuppressive therapy within 60, 30, 21 or 14 days following administration of the population of the genetically modified DNT cells.

In one embodiment, cytokines produced by the population of genetically modified DNT cells stimulate a lower level of production of IL-1β and/or IL-6 by monocytes relative to cytokines produced by genetically modified T_{conv} cells. In one embodiment, the genetically modified DNT cells do not induce cytokine release syndrome (CRS) or induce less CRS relative to genetically modified T_{conv} cells, such as CAR-T_{conv} cells.

In one embodiment, the genetically modified DNT cells are not genetically modified to reduce or eliminate expression of one or more genes selected from genes encoding for HLA, endogenous T cell receptor, CD7 and CD52.

In one embodiment, the target antigen is CD4 or CD8. In one embodiment, a population of genetically modified DNT cells that bind to, for example, a CD4 or CD8 target antigen, do not induce fratricide or induce less fratricide relative to a population of conventional T cells or CAR4 or CAR8 transduced conventional T cells.

In one embodiment, the cancer is a hematological malignancy, optionally leukemia or lymphoma. In one embodiment, the cancer is Non-Hodgkin's lymphoma, acute lymphoblastic leukemia, acute myeloid leukemia, or chronic lymphocytic leukemia. In one embodiment, the cancer in the subject comprises one or more solid tumors. In one embodiment, the cancer is lung cancer.

In one embodiment, the cancer is relapsed cancer. In one embodiment, the cancer is relapsed cancer in a subject who previously received treatment with a population of CAR-T_{conv} (CD4+/CD8+) cells. In one embodiment, the cancer exhibits a heterogeneous expression of the target antigen.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIGS. 1a-d show that ex vivo expanded DNT cells can be transduced with CAR without affecting their phenotype and expansion profile. Ex vivo expanded DNT cells were either non-transduced (NT-DNT) or transduced with CAR-19 (CAR19-DNT). 1a) Histograms show Protein L and streptavidin staining on CAR19-DNT (empty) or NT-DNT (filled). The number represents the percentage of CAR19 expressing DNT cells. Dot graph shows the summary of CAR19 transduction rate on DNT cells derived from 5 different individuals. Each dot represents a transduction rate for DNTs expanded from a single donor. Horizontal line represents the mean, and the error bar represents SD. 1b) CAR19 expression on DNT cells is maintained over time. CAR19 expression level was determined by Protein L staining on CAR19-transduced DNT cells obtained from two different donors (Donor A and Donor B) on day 0, 3, 6, 9, and 12 post-transduction. Each line represents a different donor. 1c) Ex vivo expanded DNT cells with or without CAR19-transduction were stained with anti-CD3, -CD4, and -CD8 antibodies. Figures shown are gated on CD3+ cells. Left panel shows NT-DNT cells and right panel shows CAR19-transduced DNT cells. 1d) Expansion profile of NT- and CAR19-transduced DNT cells from two different donors.

FIGS. 2a-c show that CAR19-transduction enhances anti-leukemic activity of DNT cells against CD19+ B-ALL targets in vitro. 2a and 2b) Percent specific killing induced by DNT cells against CD19+ B-ALL cell line NALM-6 after four-hour incubation at the indicated effector-to-target ratio (a) or against CD19+ primary B-ALL blasts from 5 patients for two-hours hours at 4-to-1 effector-to-target ratio (b). 2c) IFNγ level in supernatants from NT- or CAR19-DNT cells after co-culture with NALM-6 measured using ELISA.

FIGS. 3a-d show that CAR19-transduction enhances anti-leukemic activity of DNT cells against CD19+ B-ALL targets in a xenograft model. Subleathally irradiated NSG mice were inoculated with 10⁶ NALM-6 cells on day 0 followed by intravenous administration of various doses of CAR19-DNT cells (0.33×10⁶, 10⁶, or 3×10⁶ cells per mouse) or vehicle control on day 3. Bone marrow samples were collected 3 weeks post NALM-6 injection, stained with anti-human CD10 and analyzed by flow cytometry for detection of NALM6. 3a and 3b) Frequency of NALM-6 in the bone marrows of NALM-6-engrafted NSG mice. 3a. Each dot represents NALM-6 engraftment level in each mouse. Horizontal bars represent the mean of each group. Error bar represents SD. One-way ANOVA was used to determine the difference between different treatment groups. 3b. Flow cytometry dot plots show the frequency of NALM-6 in each mouse, gated on human CD10+ cells. Overall sickness score (3c) and survival (3d) of NALM-6-engrafted NSG mice treated with vehicle (dotted line) or CAR19-DNTs (solid line; 3×10⁶ cells per mouse).

FIGS. 4a and 4b show that CAR19-DNTs induce superior anti-leukemic activity against B cell lymophoblast cell line, Daudi, in a xenograft model. Sublethally irradiated NSG mice were inoculated with 10⁶ Daudi cells on day 0 followed by intravenous administration of NT-DNT cells or CAR19-DNT cells (3×10⁶ cells per mouse) on day 3. 4a) Bone marrow samples were collected 48 days post Daudi injection, stained with anti-human CD20 and analyzed by flow cytometry for detection of Daudi. Each dot represents Daudi engraftment level in each mouse. Horizontal bars represent the mean of each group. Error bar represents SD. Student's t-test was used to determine the difference between different treatment groups. 4b) Body weight change were monitored to assess for mouse fitness. Each dot represents the mean of each group. Error bar represents SEM. Two-way ANOVA was used to determine the difference between two groups.

FIGS. 5a-c show that CAR19-DNTs can kill CD19low NALM-6. 5a) representative flow plots of CD19 expression levels by CD10+ NALM-6 cells obtained from the bone marrow of untreated or CAR19-DNT cell treated mice. Numbers represent the frequency of CD19+ cells. 5b) summary of CD19 expression level by NALM-6 measured at humane end point. Horizontal bars represent the mean for each group, and error bar represents SD. Each dot represents an individual mouse. 5c) NALM-6 cells were isolated from CAR19-DNT treated (CD19low) or BPS treated (CD19high) mice and used as targets for CAR19-DNTs in a 2-hr in vitro cytotoxicity assay. Comparable degree of cytotoxicity was seen.

FIGS. 6a-c show that CAR19-DNTs and CAR19-T_{conv} cells have comparable degree of anti-leukemic activity against CD19+ B-ALL in vitro and in vivo. 6a) CAR19-DNTs or CAR19-T_{conv} cells were co-cultured with NALM-6 at the indicated effector-to-target ratio for 2 hours. % specific killing of NALM-6 was determined by flow cytometry. 6b) IFNγ level in the supernatants from CAR19-DNTs or -CAR19 T_{conv} cells co-cultured with NALM-6 measured by using ELISA. 6c) Frequency of NALM-6 in bone marrows of NALM-6-engrafted NSG mice intravenously administered with 3×106 CAR19-DNT cells, CAR-T_{conv} cells or vehicle control three-weeks post NALM-6 injection determined by flow cytometry.

FIGS. 7a-c show that CAR19-DNTs can target CD19 negative leukemic cells via endogenous anti-leukemic activity. 7a) Two-hour in vitro killing assay conducted using CAR19-DNTs (filled symbol) or NT-DNTs (empty symbol) against CD19-negative leukemia cell line, OCI-AML3, at the indicated effector-to-target ratio show that both CAR19-DNT and NT-DNT can effectively target OCI-AML3 at a comparable level. 7b) Two-hour in vitro killing assay conducted using DNT cells against a primary CD19-negative B-ALL sample obtained from a patient whose disease relapsed after CD19-CAR T_{conv} cell therapy at various effector-to-target ratio, demonstrating endogenous anti-leukemic activity of DNTs against blasts resistant to CAR19-T_{conv} cells. 7c) NT-DNTs or CAR19-DNTs were used as effectors against CD19+ lymphoma cell line, Daudi, during a two-hour in vitro killing assay at various DNT to Daudi ratios as indicated.

FIGS. 8a-b show that NT-DNT, but not NT-T_{conv} cells, mediate anti-leukemic activity against B-ALL, and CAR19-transduction enhances anti-leukemic activity against B-ALL for both T_{conv} and DNT cells. NALM-6 cells were cultured with or without NT-DNT cells, CAR19-DNT cells, NT-T_{conv} cells, or CAR19-T_{conv} cells for 2 or 5 days at 1:1 DNT-to-NALM-6 ratio. Absolute number (8a), and relative change in frequency (8b) of viable NALM-6 cells were determined. FIG. 8c shows representative flow cytometry plots from each co-culture on different days. One-way (a) or two-way (b) ANOVA tests were used for statistical analysis. ns—not significant; *p<0.05; **p<0.01; ***p<0.001; ****p<0.0001.

FIGS. 9a-f shows that CAR19-DNTs do not develop alloreactivity against normal cells and avoid alloreactivity of allogeneic effectors by suppressing them. 9a. Schematic diagram of mixed lymphocyte reaction (MLR). CAR19-DNT and CAR19-T_{conv} cells were primed with irradiated allogeneic PBMCs for 6 days, and then were used as effector cells against the same allogeneic PBMCs in a subsequent overnight killing assay. 9b. Percentage specific killing of allogeneic PBMC mediated by CAR19-T_{conv} cells (filled symbol) and CAR19-DNT cells (empty symbols) at various effector-to-target ratio, demonstrating that whereas CAR19-T_{conv} cells induce alloreactivity, CAR19-DNT cells do not. 9c) Schematic diagram of MLR conducted to determine whether CAR19-T_{conv} cells or -DNTs induce alloreactivity of allogeneic PBMCs. Allogeneic PBMCs were co-cultured with CAR19-T_{conv} cells or CAR19-DNT cells derived from another donor for 6 days. Subsequently, PBMC-derived CD8+ T cells were isolated and used as effector cells against the same T_{conv} cells or DNT cells used for co-culture in an overnight in vitro killing assay. 9d) Reduction in number of remaining viable CAR-T_{conv} cells (filled symbol) or -DNT cells (empty symbol) after an overnight culture with CD8+ T cells, as described above, suggesting that in contrast to allogeneic CAR19-T_{conv} cells, allogeneic CAR19-DNTs do not induce alloreactivity of the recipient immune cells and may evade host-versus-graft rejection. 9e) Schematic diagram of an assay used to determine the degree of cytotoxicity of alloreactive CD8+ T cells when primed with alloantigen in the presence or absence of DNTs. Allogeneic CD8+ T cells were stimulated with irradiated PBMCs with or without DNTs for four days. Subsequently, CD8+ T cells were isolated and used as effector cells against the same allogeneic cells initially used for stimulation. 9f) Percent killing of allogeneic cells by CD8+ T cells stimulated in the presence or absence of DNTs at various effector to target ratio is shown. **p<0.01; ****p<0.0001.

FIGS. 10a-d show that CAR19-DNT cells do not induce xenogeneic GvHD while CAR19-T_{conv} cells do. Naïve-NSG mice were infused with 5×10⁶ cells of CAR19-transduced DNT or T_{conv} cells. Change in mouse body weight (10a), overall sickness score (10b), and survival of mice in each group were monitored. 10d) Mouse liver tissue was Hematoxylin and eosin stained to assess for tissue damage. Two-way ANOVA test (10a and 10b) and Mantel-Cox Cox test (10c) were used for statistical analysis. n.s.—not significant; **p<0.01; ***p<0.001; ****p<0.0001

FIGS. 11a-b show that CAR19-DNTs may cause less severe CRS. NT- or CAR19-transduced DNT cells or T_{conv} cells were cultured with NALM-6 at a 5:1 (a) or 1:1 (b) T cell to NALM-6 ratio for 3 days. Supernatants collected from the co-culture were added to a macrophage-like cell line, mTHP-1 (11a), or a monocytic cell line, THP-1 (11b). IL-1β (11a) and IL-6 (11b) levels produced by mTHP-1 and THP-1, respectively, were determined 3-4 days after culture by ELISA. Two-way ANOVA test was used for statistical analysis. ns—not significant; *p<0.05; ****p<0.0001.

FIG. 12 shows that CAR19-DNT cells retain their anti-tumor function after cryopreservation. Two-hours in vitro killing assay was conducted against NALM-6 using fresh and cryopreserved CAR19-DNTs, showing that CAR19-DNTs that were cryopreserved for over a month are as potent as freshly expanded CAR19-DNTs in mediating cytotoxicity against CD19+ leukemia targets.

FIG. 13 shows that CAR19-transduced DNT cells function in donor-independent manner. DNT cells obtained from three different donors were transduced with CAR19 and used as effector cells against NALM-6. Each line represents CAR19-transduced DNT cells from each donor and showed similar killing of NALM-6 cells (Donor A, B, or C)

FIGS. 14a-c show that DNTs can be used as a platform to target an array of cancer types. 14a) In vitro killing assay conducted using NT-DNTs or CAR19-DNTs as effectors against lung cancer cell line, A549, wildtype or genetically modified to express CD19 at 1 to 1 effector to cancer cell ratio overnight. 14b) In vitro killing assay conducted using NT-DNTs or CAR19-DNTs against lung cancer cell line, H460, wildtype or genetically modified to express CD19 at 1 to 1 effector to cancer cell ratio overnight. 14c) NT-DNTs or CAR19-DNTs were co-cultured with A549 or CD19-transduced A549 at 1:1 DNT:A549 ratio for 1 day. IFNγ in the supernatants collected from each group using ELISA.

FIGS. 15a-b show that CAR-DNT cells exhibit potent anti-tumor activity against solid tumor in a lung cancer xenograft model. NSG mice were subcutaneously infused with CD19-transduced A549 (10⁶/mouse) on day 0. On day 11, each mouse was untreated or treated with 5×10⁶ NT-DNT or CAR-19+ DNT cells via peri-tumoral injection. Subsequently, tumor volumes were monitored every 2-4 days (15a), and the tumor weight was measured at the end of the study (15b). Two-way (15a) or One-way (15b) ANOVA test was used for statistical analysis. n.s—not significant; *p<0.05; ***p<0.001; ****p<0.0001

FIGS. 16a-b show that DNTs can be used as a platform for different CAR constructs to target cancers that express antigens other than CD19 and as a carrier for CD4-CAR without fratricide. 16a) Comparable expansion fold of NT-DNTs (●) and CD4-CAR (CAR4; ▪)-transduced DNTs support lack of fratricide in manufacturing of CAR4-DNTs. 16b) NT-DNTs (filled symbols) or DNTs transduced with CD4-CAR (CAR4-DNT; empty symbols) were used as effector cells against CD4+ acute lymphocytic T cell leukemia line CCRF-CEM during a two-hour killing assay at various DNT to CCRF-CEM ratios as indicated.

FIGS. 17a-b show the antigen specificity of CAR4-DNTs. Healthy donor-derived allogeneic PBMCs were co-cultured with NT or empty viral vector-(EV), or CAR19−, or CAR4-transduced DNT cells at various DNT:PBMC ratio. The % specific killing was measured against CD4+ (17a) and CD4− (17b) PBMCs. Two-way ANOVA tests was used for statistical analysis. n.s—not significant; ****p<0.0001

FIGS. 18a-b show that DNTs can be transduced with CAR4 without fratricide. T_{conv} cells or DNT cells were non-transduced or transduced with CAR4. On day 4 post transduction, the relative number (18a) and the composition of CD4+, CD8+ and CD4− CD8− (DN; 18b) were compared.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

As used herein, the term “cancer” refers to one of a group of diseases caused by the uncontrolled, abnormal growth of cells that can spread to adjoining tissues or other parts of the body. Cancer cells can form a solid tumor, in which the cancer cells are massed together, or exist as dispersed cells, as in a hematological cancer such as leukemia.

The term “cancer cell” refers a cell characterized by uncontrolled, abnormal growth and the ability to invade another tissue or a cell derived from such a cell. Cancer cells include, for example, a primary cancer cell obtained from a patient with cancer or cell line derived from such a cell. In one embodiment, the cancer cell is a hematological cancer cell such as a leukemic cell or a lymphoma cell.

The term “subject” as used herein includes all members of the animal kingdom including mammals, and suitably refers to humans. Optionally, the term “subject” includes mammals that have been diagnosed with cancer or are in remission. In one embodiment, the term “subject” refers to a human having, or suspected of having, cancer.

In one embodiment, the methods and uses described herein provide for the treatment of cancer. The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. In one embodiment, treatment methods comprise administering to a subject a therapeutically effective amount of CAR-DNT cells as described herein and optionally consists of a single administration, or alternatively comprises a series of administrations.

As used herein, the phrase “effective amount” or “therapeutically effective amount” means an amount effective, at dosages and for periods of time necessary to achieve the desired result. For example in the context or treating cancer, an effective amount is an amount that for example induces remission, reduces tumor burden, and/or prevents tumor spread or growth of cancer cells compared to the response obtained without administration of the compound. Effective amounts may vary according to factors such as the disease state, age, sex and weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

In one embodiment, the methods, uses and compositions described herein involve the production, administration, or use of DNT cells that have been genetically modified to bind to one or more target antigen(s). For example, in one embodiment, the methods, uses and compositions described herein involve the production, administration, or use of CAR-DNT cells or CAR-DNTs. As used herein, “CAR-DNT cells” or “CAR-DNTs” refer to double negative T-cells (“DNT cells” or “DNTs”) that have been modified to express one or more chimeric antigen receptor (CAR) molecules. Such CAR-DNT cells may be described as being derived from DNT cells.

DNTs exhibit a number of characteristics that distinguish them from other kinds of T cells. In one embodiment, the DNTs do not express CD4 or CD8. In one embodiment, the DNTs expanded for 10-20 days express CD3-TCR complex and do not express CD4 and CD8. In one embodiment, the DNTs are CD4−, CD8−, CD3+, γδ-TCR+ and/or αβ-TcR+. In one embodiment, the DNTs are CD4−, CD8−, CD3+, γδ-TCR+ and αβ-TcR+. In one embodiment, expanded DNTs may be CD11a+, CD18+, CD10−, and/or TCR Vα24-Jα18−. In one embodiment, expanded DNTs may be CD49d+, CD45+, CD58+ CD147+ CD98+ CD43+ CD66b− CD35− CD36− and/or CD103−.

DNTs may be obtained using technologies known in the art such as, but not limited to, fluorescent activated cell sorting (FACS). Methods for producing and/or expanding DNT cells are also described in WO2007056854 as well as WO2016023134, which are hereby incorporated by reference.

As used herein, the term “autologous” refers to cells originally obtained from a subject who is the intended recipient of said cells. In one embodiment the DNT cells or the population of DNT cells described herein comprises or consist of autologous cells.

As used herein, the term “allogenic” refers to cells originally obtained from a subject who is a different individual than the intended recipient of said cells, but who is of the same species as the recipient. Optionally, allogenic cells may be cells from a cell culture. In a one embodiment, the DNTs are allogenic cells obtained from a healthy donor. As used herein the terms “healthy donor” (“HD”) refer to one or more subjects without cancer. In one embodiment, the healthy donor is a subject with no detectable cancer cells, such as a subject with no detectable leukemic cells. In one embodiment, the genetically modified DNT cells described herein are allogenic cells, optionally from one or more healthy donors. In one embodiment, a population of genetically modified DNT cells as described comprise or consists of allogenic cells, optionally from one or more healthy donors.

As used herein, the term “CAR” refers to a chimeric antigen receptor. In one embodiment, the CAR molecule comprises an extracellular antigen binding domain, a hinge region, a transmembrane domain, and one or more intracellular domains such as a co-stimulatory signaling domain and/or a CD3 zeta domain. The antigen binding domain may bind any suitable antigen, for example an antigen enriched or preferentially expressed on the surface of a cancer cell. In one embodiment, the antigen binding domain binds CD19. In one embodiment, the antigen binding domain binds CD4.

In one embodiment, the genetically modified DNT cells are derived from DNT cells by genetically modifying the cells to express a protein on the surface of the DNT cell that binds to a target antigen. For example, in one embodiment, CAR-DNT cells are derived from DNT cells by modifying DNT cells to express one or more CAR molecules. DNT cells may be modified to express one or more CAR molecules by any suitable technique. Optionally, DNTs may be genetically modified by transduction with a suitable expression vector, plasmid or mRNA.

In one embodiment, the genetically modified DNTs may be cryopreserved prior to administration or use in a subject. As used herein, “cryopreservation” refers to the process by which cells, for example genetically modified DNTs such as CAR-DNTs, are preserved by cooling to very low temperatures. Such low temperatures may be in the range of −70° C. to −90° C. using a −80° C. freezer or solid carbon dioxide, or −196° C. using liquid nitrogen and are utilized to slow/stop any enzymatic or chemical activity which might cause damage to the cells. Cryopreservation methods seek to reach low temperatures without causing additional damage caused by the formation of intracellular ice crystals during freezing. Alternatively, freshly expanded DNT cells without cryopreservation may be genetically modified and used or adminstered as described herein.

As used herein, “immunosuppressive therapy” refers to the administration or use of one or more pharmaceutical agents to suppress the immune system of a subject in order to prevent or diminish graft-versus-host disease (GvHD), host-versus-graft rejection, and/or alloreactivity. Examples of immunosuppressive therapies include, but are not limited to, alemtuzumab, calcineurin inhibitors such as cyclosporin A (CSA), tacrolimus (TAC), target of rapamycin (TOR) inhibitors such as sirolimus (SIR) and/or antiproliferatives such as mycophenolate mofetil (MMF).

As used herein, “cytokine release syndrome” or “CRS” refers to a condition that may occur after immunotherapy characterized by a large, rapid, and systemic release of inflammatory cytokines by the infused products and the host immune cells affected by the immunotherapy resulting in a systemic inflammatory response.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

It must be noted that as used herein and in the appended claims, the singular forms “a”, “an”, and “the” include plural references unless the context clearly dictates otherwise.

All numerical values within the detailed description and the claims herein are modified by “about” or “approximately” the indicated value, and take into account experimental error and variations that would be expected by a person having ordinary skill in the art.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “about” as used herein means plus or minus 0.1 to 50%, 5-50%, or 10-40%, 10-20%, 10%-15%, preferably 5-10%, most preferably about 5% of the number to which reference is being made

It should also be understood that, in certain methods described herein that include more than one step or act, the order of the steps or acts of the method is not necessarily limited to the order in which the steps or acts of the method are recited unless the context indicates otherwise.

Although any methods and materials similar or equivalent to those described herein can also be used in the practice or testing of the present disclosure, the preferred methods and materials are now described.

### II. Products and Compositions

In one aspect, there is provided a double negative T (DNT) cell that has been genetically modified to bind to one or more target antigen(s). In one embodiment, the DNT cells is genetically modified to express a nucleic acid molecule encoding a chimeric antigen receptor (CAR) that binds to a specific target antigen.

Any suitable method can be used to modify a DNT cell to express a nucleic acid encoding a protein that binds to the target antigen such as, but not limited to, a CAR. As shown in the Examples, the modified DNT cell can be generated by transduction with a vector, plasmid or mRNA comprising a sequence encoding for the CAR. Accordingly, in one embodiment, the modified DNT cell is generated by transduction with a vector comprising a nucleic acid molecule encoding a CAR or another suitable protein that binds to the target antigen.

The target antigen may be any suitable antigen such as a target that is expressed on the surface of a cancer cell. Suitable antigens include, but are not limited to CD4, CD33, CD19, CD20, CD123 LeY, Mesothelin, EGFR, ROR1, EpCam, MUC1, HER1/2, MET/HGF, neoantigens (driver, non-driver), MAGE family and NY-ESO-1. In one embodiment, the target antigen is CD19. In one embodiment, the target antigen is CD4

In one embodiment, the CAR comprises an extracellular binding domain, a hinge region, a transmembrane domain and/or an intracellular signaling domain. In one embodiment, the extracellular binding domain of the CAR binds to a suitable target antigen. For example, the CAR may comprise an extracellular antigen binding domain that binds to a target antigen expressed on a cancer cell.

As shown in the Examples, the genetically modified DNT cells described herein maintain activity after cryopreservation. Accordingly, in one embodiment the genetically modified DNT cell has been frozen or cryopreserved. For example, in one embodiment, populations of allogenic genetically modified DNT cells as described herein may be expanded and modified ex vivo and then cryo-preserved in order to produce an off-the-shelf cellular therapy suitable for clinical use. In one embodiment, the cells are frozen as a temperature less than −20° C., less than 50° C., less than 60° C., between −20 and −196° C., between −70° C. and −196° C. or between −70° C. and −90° C.

The modified DNT cells according to the present disclosure may be provided in the form of a composition. For example, in one embodiment there is provided a composition comprising a population of modified DNT cells described herein, and a pharmaceutically acceptable carrier. The CAR-DNTs may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The term “pharmaceutically acceptable” means compatible with the treatment of animals, in particular, humans. Also provided are kits comprising genetically modified DNTs as described herein, along with suitable container or packaging and/or instructions for the use thereof, such as for the treatment of cancer in a subject.

Also provided are populations of modified DNT cells as described herein. In one embodiment, there is provided a population of allogenic genetically modified DNTs generated from one or more healthy donors. In one embodiment, the population of allogenic genetically modified DNTs cells does not induce graft-versus-host disease (GvHD) in a subject. In one embodiment, the population of allogenic genetically modified DNT cells induces less GvHD in a subject relative to conventional CAR-T cells (CAR-T_{conv} cells).

In one embodiment, the population of allogenic genetically modified DNTs cells avoids or suppresses host-versus-graft rejection in a subject. In one embodiment, the population of CAR-DNT cells avoids or suppresses HvG rejection in a subject relative to CAR-T_{conv} cells. In one embodiment, there is provided a population of CAR4-DNTs. In one embodiment, the population of CAR4-DNTs does not induce fratricide. Also provided is a population of CARS-DNTs.

In one embodiment, the genetically modified DNT cells described herein do not require modifications in order to avoid HvG rejections or GvHD. For example, in one embodiment, the genetically modified DNT cells described herein, optionally CAR-DNTS such as CAR4-DNTs, are not genetically modified to reduce or eliminate expression of one or more genes selected from genes encoding for HLA (optionally class I or class II), T cell receptor, CD7 or CD52.

### III. Methods and Uses

As shown in the Examples, the genetically modified DNT cells have been demonstrated to enhance cytotoxicity against cancer cells including a B-cell acute lymphoblastic leukemia (B-ALL) cell line (NALM-6) as well as patient ALL blasts. CAR19-DNTs were also shown to be effective at prolonging survival in a murine NALM-6 xenograft model, and mice receiving CAR19-DNT treatment exhibited a superior overall health score relative to controls. Notably, CAR19-DNTs also exhibit increased cytotoxic activity against CD19+ lung cancer cell lines relative to non-transduced controls. CAR-DNTs have also been shown not to induce off-tumor alloreactivity in contrast to conventional CAR19 T cells. Notably, while genetic modification of T_{conv} cells may increase host v. graft (HvG) rejection due to foreign peptides, the modified DNTs did not induce significant alloreactivity or exhibited less alloreactivity than conventional CAR T cells. Without being limited by theory, this may be due to the ability of DNTs to suppress T_{conv} cells mediated immune responses. DNTs transduced with anti-CD4 CAR were also generated and demonstrated to be effective against a CD4+ leukemia cell line without any signs of fratricide, indicating that DNTs are likely to be useful as a general CAR carrier or for other targeted cellular therapies.

Accordingly, in one aspect there is provided a method of treating cancer in a subject in need thereof, the method comprising administering to the subject an effective amount of a population of genetically modified DNTs that bind to a target antigen. Also provided is the use of an effective amount of a population of genetically modified DNT cells that bind to one or more target antigens for the treatment of cancer in a subject in need thereof. In one embodiment, the population of CAR-DNT cells comprises of or consists of cells that are CD4−, CD8−, CD3+, γδ-TCR+ and/or αβ-TcR+. In one embodiment, the target antigen is expressed on the surface of a cancer cell. In one embodiment, the genetically modified DNTs are chimeric antigen receptor (CAR)-double negative T (DNT) cells.

The population of genetically modified DNT cells for use in the methods or uses described herein can be derived from one or more suitable donors. Suitable donors include the subject being treated or one or more donors of the same species as the subject being treated. Accordingly, in one embodiment, the population of genetically modified-DNT cells comprises or consists of autologous cells. In one embodiment the population of genetically modified DNT cells comprises or consists of allogenic cells. Optionally the population of genetically modified DNT cells comprises or consists of allogenic cells from one or more healthy donors. In one embodiment, the population of CAR-DNT cells comprises or consists of genetically modified DNTs from one or more donors that are cryopreserved prior to their use or administration for the treatment of cancer.

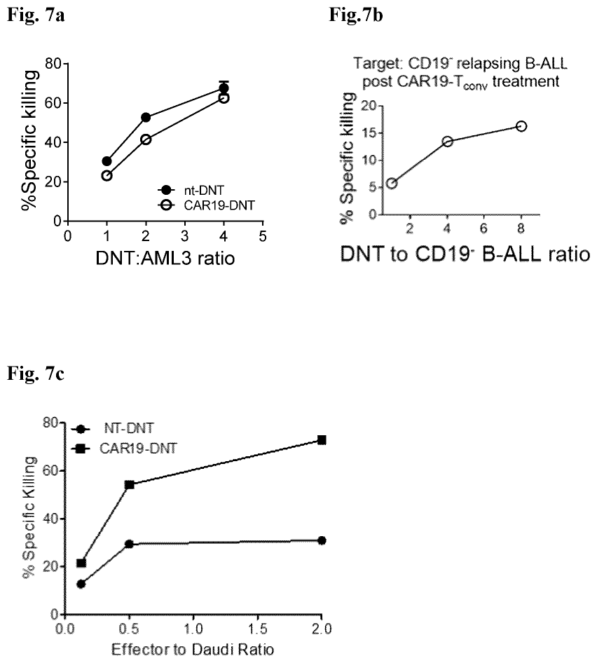

Currently available allogeneic CAR-immune cell therapies involve additional genetic modification to knock-out HLA on allogeneic CAR-T cells or deplete CD52+ recipient T cells to avoid HvG rejection (Zhao et al. 2018). Alternatively, repeated administration of immunosuppressants are used to allow multiple infusions of allogeneic CAR products. However, these will increase the cost and complexity of product manufacturing or hamper recipient immune system against infections. As shown in FIG. 7, allogenic CAR-DNT cells as described herein may co-persist with conventional T cells without developing alloreactivity and/or develop less alloreactivity relative to conventional (CD4+/CD8+) CAR T cells. In one embodiment, the allogeneic genetically modified DNTs provided herein are not genetically modified to avoid HvG rejection. In one embodiment, the genetically modified DNTs are not modified to reduce or eliminate expression of one or more genes selected from genes encoding for HLA (optionally class I or class II), endogenous T cell receptor, CD7 or CD52.

In one embodiment, allogeneic genetically modified DNTs do not cause graft-versus host disease when administered or used in a subject. In one embodiment, allogeneic genetically modified DNTs avoid host-versus-graft allo-rejection. In one embodiment, the allogenic genetically modified DNTs are resistant to host-versus-graft allo-rejection relative to conventional (CD4+/CD8+) CAR T cells, In one embodiment, the allogeneic genetically modified DNTs avoid host-versus-graft allo-rejection by suppressing alloreactive T cells, thereby can be used without the need for additional immunosuppressive therapy, after standard lymphodepletion preconditioning.

In one embodiment, the subject receives lymphodepletion chemotherapy prior to administration of the population of genetically modified DNT cells. Lymphodepletion preconditioning is believed to create space and favorable homeostatic cytokine environment in the subject for the expansion and growth of adoptively transferred lymphocytes in general. For example, in one embodiment lymphodepletion using chemotherapy (e.g. fludarabine+cyclophosphamide) may be used before the administration of T cell therapy including DNTs. Lymphodepletion preconditioning typically lasts for a few weeks, unlike longer-term immunosuppression that may be used concurrently or after the administration of cellular therapies to help avoid or reduce alloreactivity such as GvHD or HvG rejection.

In one embodiment, the subject does not receive immunosuppressive therapy concurrently, or after the administration or use of genetically modified DNT cells for the treatment of cancer. In one embodiment, no additional immunosuppressive agents such as alemtuzumab are required in the lymphodepletion chemotherapy preconditioned subject for suppressing or reducing alloreactivity or HvG to the allogeneic genetically modified DNT cells infused. For example, in one embodiment, the subject does not receive additional immunosuppressive therapy within 60, 30, 21, 14, 0 or −7 days following administration of the population of genetically modified DNT cells.

In one embodiment, while conventional allogeneic CD4+/CD8+ T cells are susceptible to host-versus-graft (HvG) rejection and additional immunosuppressive therapies such as alemtuzumab is needed to suppress HvG, the population of genetically modified DNT cells avoid or suppress HvG without the need of additional immunosuppressive therapies such as alemtuzmab. For example, in one embodiment the allogeneic genetically modified DNT cells persist in the lymphodepletion preconditioned subject for longer than 2 weeks, 3 weeks, 4 weeks, 6 weeks, or 8 weeks without additional immunosuppressive therapies to suppress HvG. In one embodiment, genetically modified DNT cells may be detected in a biological sample from the subject 2 weeks, 3 weeks, 4 weeks, 6 weeks, or 8 weeks after the administration or use of the cells in the subject.

In one embodiment, multiple doses of allogeneic genetically modified DNT cells can be infused into a patient without additional manipulation to the cells or to patients. For example, in embodiment the allogeneic genetically modified DNT cells can be re-infused into a patient within about 1 week, 2 weeks, 3 weeks, and/or 4 weeks after the initial infusion.

Genetically modified DNT cells that bind to one or more target antigens suitable for use in the methods described herein may be generated by any suitable method. For example, in one embodiment, a population of CAR-DNT cells comprises DNT cells transduced with a vector, plasmid or mRNA comprising a nucleic acid sequence encoding for one or more chimeric antigen receptors.

CAR-DNT cells for use according to the methods described herein may express one or more suitable CAR molecules. In one embodiment, the CAR comprises an extracellular binding domain, a hinge region, a transmembrane domain and/or an intracellular signaling domain. The extracellular binding domain of the CAR may bind any target antigen suitable for the methods described herein. Accordingly, in one embodiment, the CAR comprises an extracellular antigen binding domain that binds to a target antigen expressed on a cancer cell in the subject. Suitable target antigens include CD4, CD33, CD19, CD20, CD123, LeY, Mesothelin, EGFR, ROR1, EpCam, MUC1, HER1/2, MET/HGF, neoantigens (driver, non-driver), MAGE family, and NY-ESO-1. In one embodiment, the target antigen is CD19. In one embodiment, the target antigen is CD4.

The genetically modified DNT cells described herein may be used in the treatment of various cancers. In one embodiment, the cancer is a hematological malignancy such as a leukemia or lymphoma. Optionally the cancer is Non-Hodgkin's lymphoma, acute lymphoblastic leukemia, acute myeloid leukemia, or chronic lymphocytic leukemia. In one embodiment, the cancer comprises one or more solid tumors including, but not limited to, lung cancer.

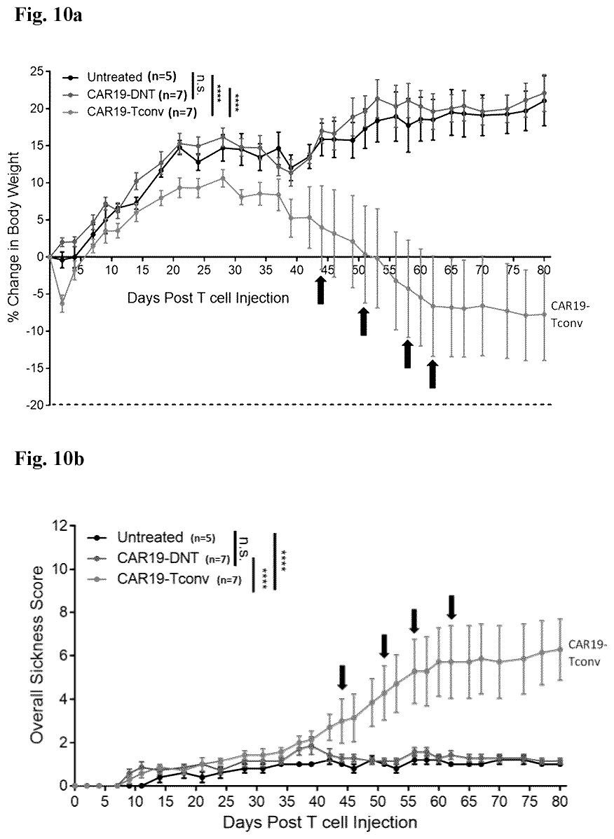

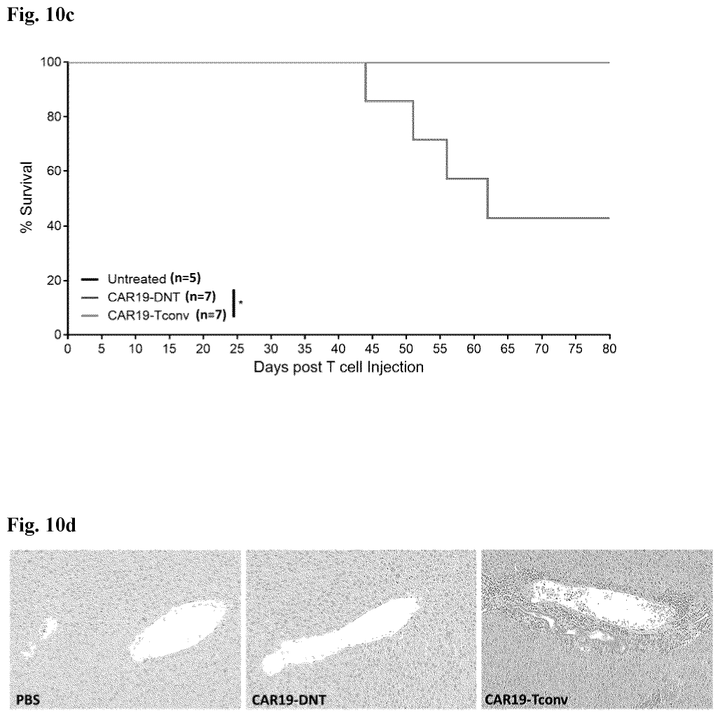

In one embodiment, there is provided a method of treating a CD4+ cancer in a subject in need thereof, the method comprising administering to the subject an effective amount of a population of DNT cells that have been genetically modified to bind to CD4. Also provided is the use of an effective amount DNTs that have been genetically modified to bind to CD4 for treating a CD4+ cancer in a subject in need thereof. In one embodiment, the DNT cells are CD4-targeting CAR-DNTs (CAR4-DNT). In one embodiment, the genetically modified DNTs are allogenic. In one embodiment, the cancer is a hematological malignancy. In one embodiment, the cancer is a T cell cancer such as T cell acute lymphoblastic leukemia (T-ALL), peripheral T cell lymphoma (PTCL) and/or cutaneous T cell lymphoma (CTCL). T cell cancers present a challenge for the use of autologous conventional CART therapy, given that cancerous T cells in the patients can contaminate the autologous T cell product used to make CART cells. Furthermore, CD4 expression in conventional T cells themselves can lead to fratricide among CD4-targeting conventional CART cells, while CAR4-DNT cell manufacturing show no signs of fratricide (FIG. 10a). As shown in FIG. 10b, allogeneic CAR4-DNT cells were shown to be cytotoxic against a CD4+ acute lymphocytic T cell leukemic cell line. Furthermore, allogenic CAR4-DNTs can avoid the issues associated with autologous T cell therapies, as they do not express CD4 and can be generated from healthy donors without contamination of cancerous T cells.

As set out in the Examples, the CAR-DNTs described herein exhibit dual CAR-targeted and CAR-independent killing of cancer cells. In one embodiment, the genetically modified DNTs described herein are for use or administration to a subject with relapsed or recurrent cancer. For example, in one embodiment, the genetically modified DNTs described herein are for use or administration to a subject with relapsing cancer after conventional CAR T cell treatment, such as conventional CAR19 T cell treatment. In one embodiment, the cancer is relapsing acute lymphoblastic leukemia, optionally B-cell acute lymphoblastic leukemia (B-ALL). In one embodiment, the cancer comprises or consists of CD19-B-ALL.

In one embodiment, the methods and uses described herein are for the treatment of cancer that exhibits a heterogeneous expression of the target antigens, such as cancer that exhibits a heterogeneous expression of CD4, CD33, CD19, CD20, CD123 and/or LeY. In one embodiment, the methods and uses described herein are for the treatment of cancer that exhibits a heterogeneous expression of Mesothelin, EGFR, ROR1, EpCam, MUC1, HER1/2, MET/HGF, neoantigens (driver, non-driver), MAGE family, and/or NY-ESO-1.

### IV. EXAMPLES

### Example 1

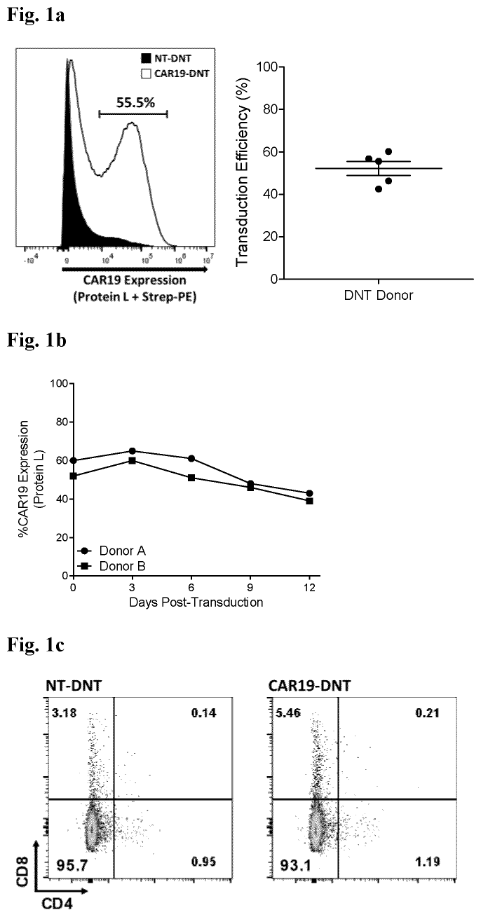

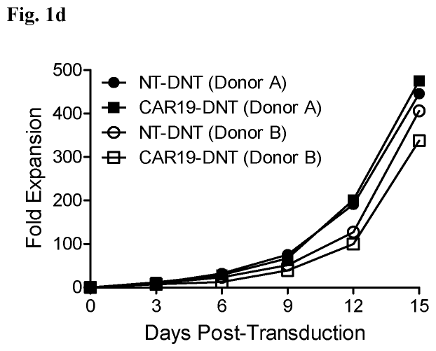

To determine if DNTs can be transduced with Chimeric Antigen Receptor (CAR), DNTs were transduced with a widely used CD19-CAR (CAR19), and its expression was determined using Protein L binding. DNTs were transduced with CAR19 with a mean transduction rate of 55.5%±7.51% (FIG. 1a), and CAR19 expression was maintained for at least 12 days (FIG. 1b). No phenotypical changes on DNTs by CAR19 transduction were seen; DNTs retained CD3-positive CD4 and CD8 double negative phenotype (FIG. 1c) and DNTs retained comparable expansion profile as those expanded without CAR19 transduction (FIG. 1d).

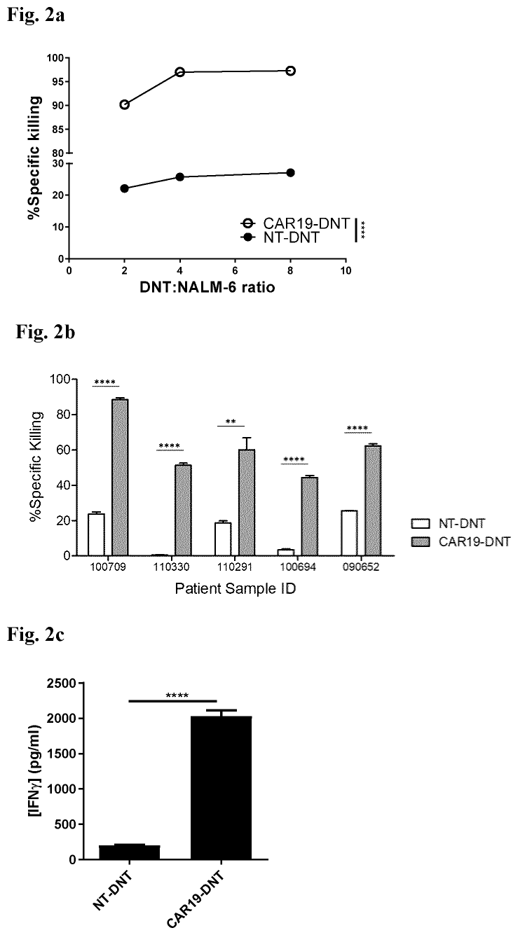

In vitro killing assay conducted using non-transduced (NT)- or CAR19-DNT cells against a CD19+B-ALL cell line NALM-6 (FIG. 2a), and five primary B-ALL patient blasts (FIG. 2b) showed that while non-transduced (NT)-DNTs can induce some degree of cytotoxicity against NALM-6 and some B-ALL patient samples (100709, 110291, and 090652), the degree of cytotoxicity is significantly enhanced by CAR19 transduction. Further, a higher level of IFNγ was detected from the supernatants after co-culture with B-ALL by CAR19+ DNT cells than that of NT-DNT cells (FIG. 2c).

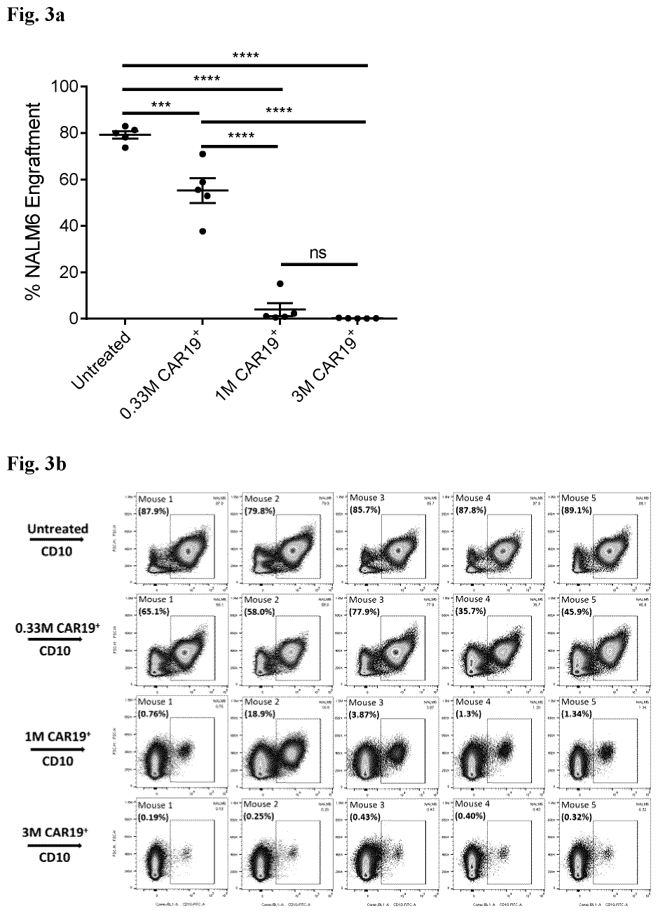

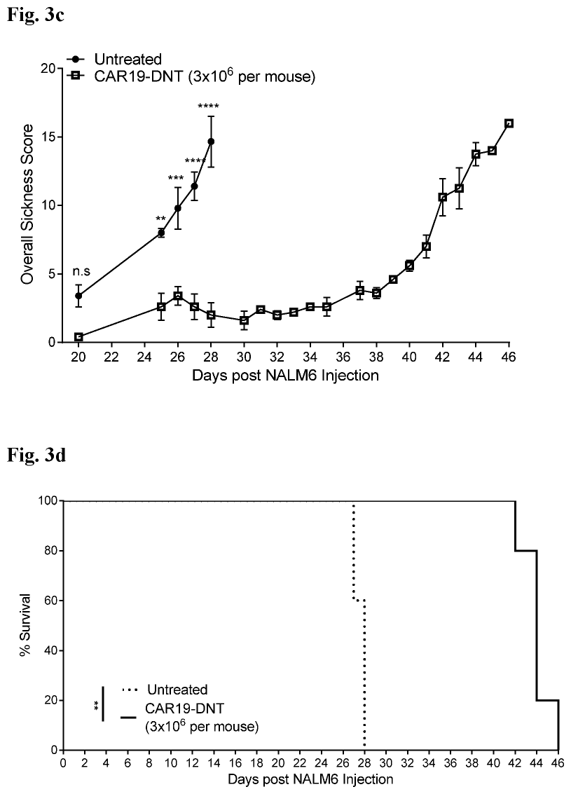

To determine the anti-leukemic activity of CAR19-DNTs in vivo, immunodeficient NSG mice engrafted with NALM-6 were untreated or treated with different numbers of CAR19-DNT cells, 0.33×10⁶, 10⁶, or 3×10⁶ cells per mouse, and the leukemia load and mice survival were compared. The NALM-6 engraftment levels in bone marrow were determined by flow cytometry. CAR19-DNT cells reduced leukemia load in a dose dependent manner, where the mean NALM-6 engraftment level was 0.19%±0.11% in mice treated with highest dose of CAR19-DNT cells as opposed to 79.2%±3.6% in the untreated group (FIGS. 3a and 3b). Further, mice treated with 3×10⁶ CAR19-DNT cells showed prolonged survival with a median survival of 44 days compared to 28 days median survival for the untreated group (FIG. 3c). Consistent with this, a significantly superior overall health score evaluated by scruffiness, arch in back, fur loss, and loss of activity was observed in CAR19-DNT treated recipients (FIG. 3d).

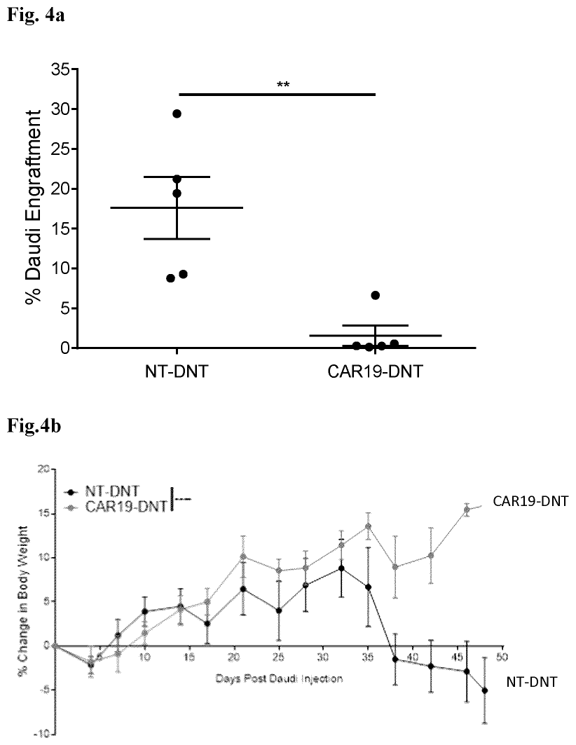

To validate our findings using a different B-ALL target, NSG mice engrafted with B cell lymphoblast line, Daudi, were treated with NT- or CAR19-DNT cells. Significantly lower levels of Daudi engraftment were observed in the bone marrow of mice treated with CAR19-DNT cells than those seen with NT-DNT treated mice (FIG. 4a). Further, significantly lower mouse body weight was observed in NT-DNT group than CAR-DNT group (FIG. 4b), suggesting superior fitness of mice treated with CAR19-DNT cells compared to those treated with NT-DNT cells.

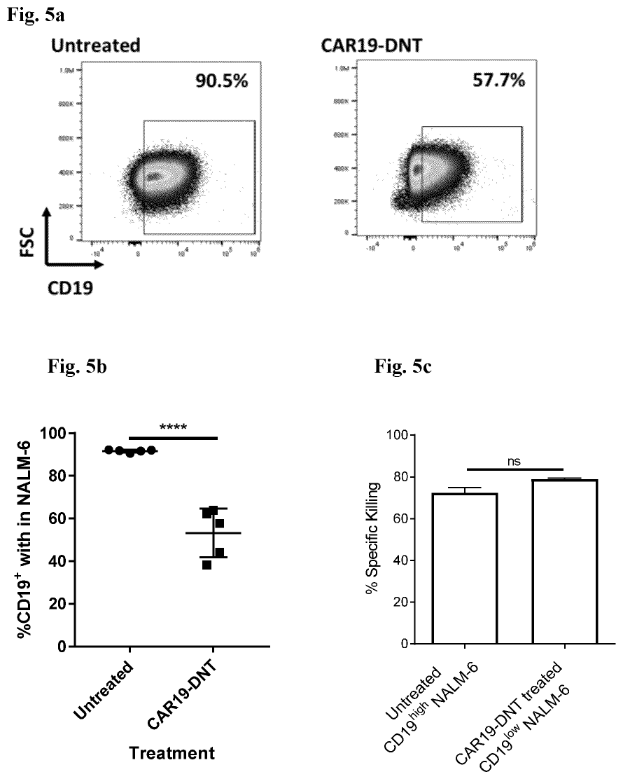

Ex vivo analysis of NALM-6 at humane endpoints showed reduced expression of CD19 by NALM-6 obtained from CAR19-DNT cell-treated group than those from the untreated (FIGS. 5a and 5b; 53.22%±5.12% versus 91.67%±0.30%), suggesting that NALM-6 may evade CAR19-DNT cell mediated anti-leukemia activity through CD19 downregulation as seen CD19− B-ALL relapse patients after CAR-T cell treatment. Surprisingly, when NALM-6 obtained from CAR19-DNT treated mice were used as targets for CAR19-DNT ex vivo, they were killed as effectively by CAR19-DNT cells as untreated NALM-6 (FIG. 5c), suggesting that additional treatment with CAR19-DNT cells may enhance the therapeutic benefits of CAR19-DNT cells.

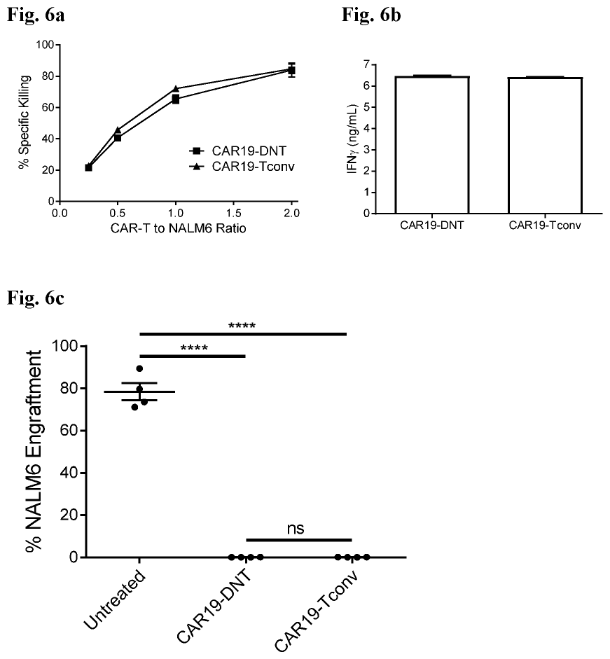

To compare the potency of CAR19-DNTs to that of CAR19-T_{conv} cells, in vitro killing assays against NALM-6 were conducted using CAR19-DNTs and CAR19-T_{conv} cells derived from same healthy donor and showed that both cell types induced comparable degree of cytotoxicity (FIG. 6a). Similarly, comparable amounts of IFNγ were produced by CAR19-DNTs and CAR19-T_{conv} after co-culture with NALM-6 (FIG. 6b). Further, treating NALM-6 engrafted NSG mice with an equal number of CAR19-DNTs and CAR19-T_{conv} cells both resulted in leukemia eradication, supporting that CAR19-DNTs induce a comparable degree of anti-leukemia activity as that of CAR19-T_{conv} cells (FIG. 6c).

Ruella et al. (Ruella, Barrett et al. 2016), showed that CAR with dual specificities can prevent B-ALL relapse by CD19 downregulation. Given that DNT cells have endogenous anti-leukemia activity mediated by NKG2D and DNAM-1, the ability of CAR19-transduced DNTs and NT-DNTs to mediate cytotoxicity towards CD19− leukemic cell lines and primary B-ALL blasts from patient relapsed after CAR19-T_{conv} cell treatment was tested. Notably, CAR19-DNTs effectively induce cytotoxicity in the absence of CD19 expression on the target cells (FIG. 7a). Interestingly, a significant degree of in vitro DNT-mediated cytotoxicity was seen against primary B-ALL blasts obtained from patients that relapsed after received conventional CD19-CAR T cell treatment in a dose-dependent manner (FIG. 7b). Further, while CAR19-DNTs showed superior cytotoxicity against CD19+ non-Hodgkin lymphoma cell line, Daudi, NT-DNTs induced significant degree of cytotoxicity against Daudi in a dose-dependent manner (FIG. 7c). These results suggest that through their dual cytotoxic function, CAR-mediated and endogenous anti-leukemic activity, CAR-DNTs have the potential to prevent antigen-negative relapse in patients, thus increasing their overall therapeutic value. Collectively, CAR19-DNTs may induce superior anti-leukemia activity than that of CAR19-T_{conv} cells by preventing immune escape through CAR-antigen downregulation and has the potentials to treat relapse patients after conventional CAR19 T cell treatment.

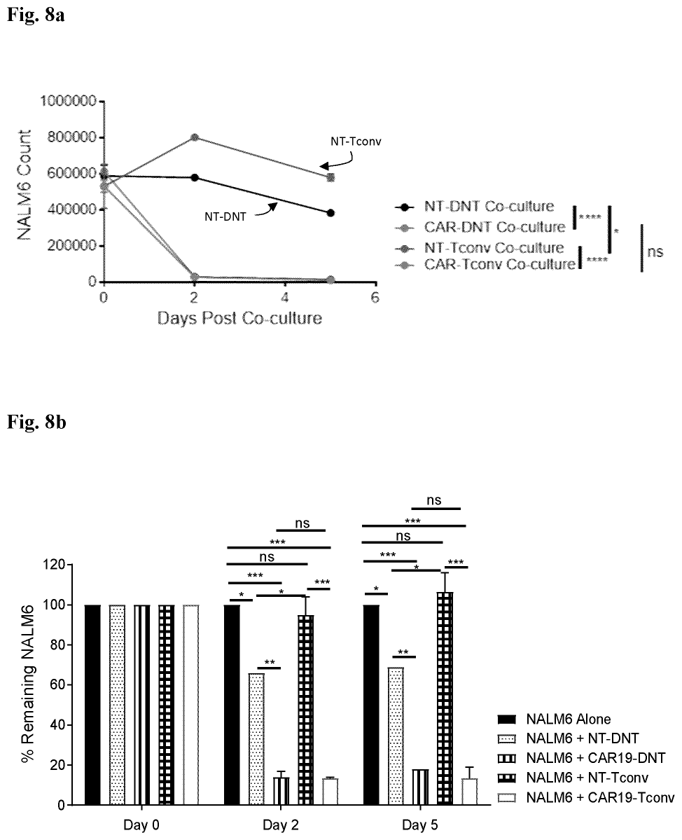

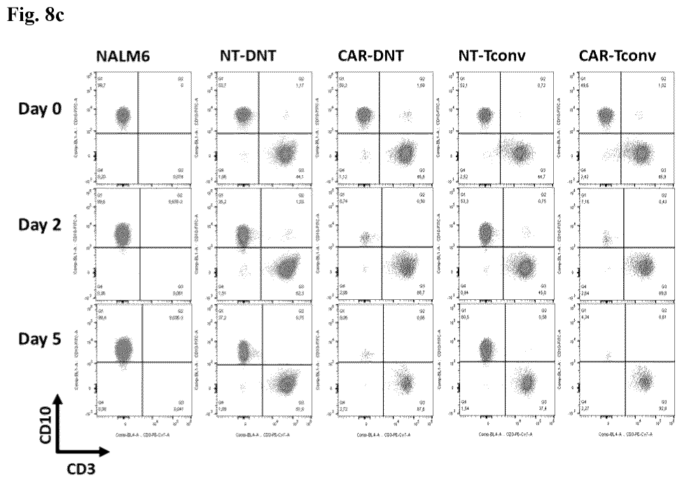

To compare the endogenous and CAR19-mediated anti-leukemic activity against B-ALL, NALM-6 cells were cultured with or without NT-DNT cells, CAR19-DNT cells, NT-T_{conv} cells, or CAR19-T_{conv} cells for 2 or 5 days. A significant lower number of NALM-6 cells in NT-DNT cell-treated culture than those treated with NT-T_{conv} cells was observed, demonstrating that DNT cells, but not T_{conv} cells, mediate endogenous anti-leukemic activity against NALM-6 (FIGS. 8a and 8b). However, a notable difference in the number of NALM-6 cells cultured between CAR19-DNT cells and CAR19-T_{conv} cells was not seen (FIGS. 8a and 8b). Similarly, marked reduction in frequency of NALM-6 cells was observed over time when co-cultured with NT-DNT cells, while co-culture with NT-T_{conv} cells did not reduce the relative NALM-6 frequency (FIG. 8c). Co-culture with both CAR19-DNT cells and CAR19-T_{conv} cells reduced the NALM-6 cell frequency below 10% in 2 days (FIG. 8c).

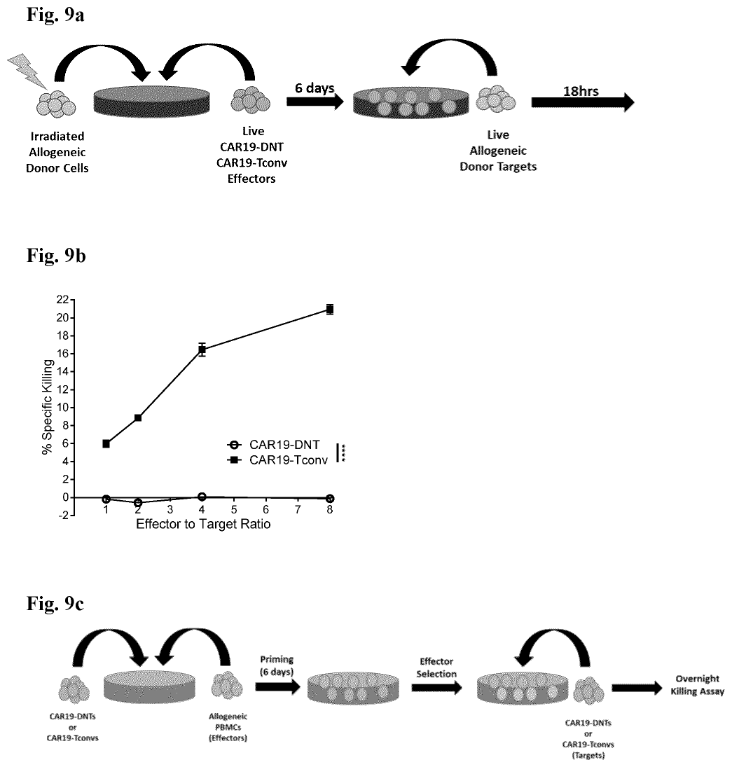

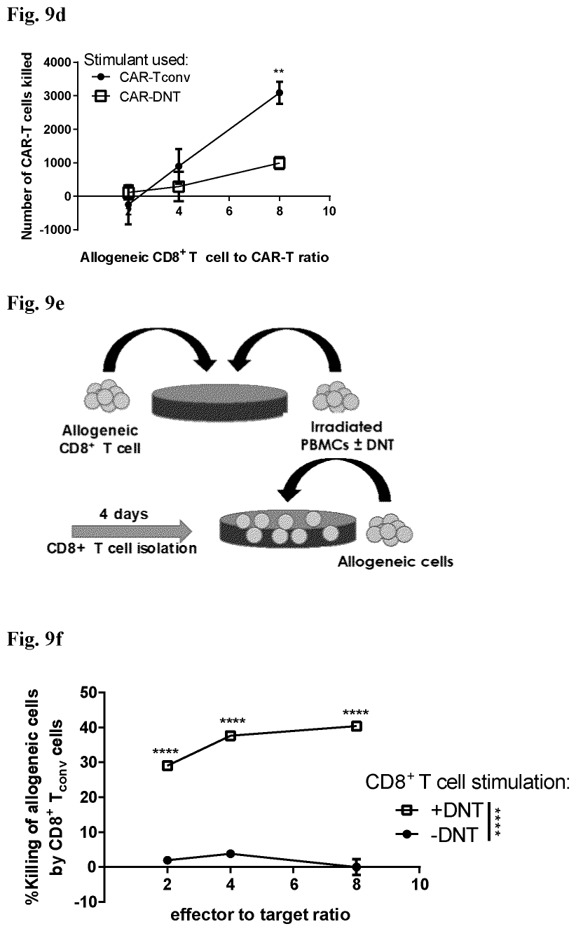

Non-genetically modified DNTs have previously been shown not to cause GvHD or induce alloreactivity. In order to use allogeneic CAR-DNTs as an off-the-shelf cellular therapy, it is important to determine whether these cells elicit GvHD and/or HvG rejection as CAR19-transduced T cells can have higher basal activation level due to increased activation intracellular domains from CARs and may induce alloreactivity as a result of foreign antigens derived from CARs. Therefore, in vitro mixed lymphocyte reaction (MLR) assays were conducted to determine the potential of CAR19-DNTs to induce allogeneic immune responses. CAR19-DNTs were stimulated with irradiated allogeneic PBMCs (FIG. 9a). In parallel, CAR19-T_{conv} cells were stimulated as a control. CAR19-DNTs and CAR19-T_{conv} cells primed with allo-antigens were then harvested and used as effectors against viable PBMCs from the same allogeneic donor. As seen with DNTs without CAR modification (NT-DNT), allo-antigen primed CAR19-DNTs developed no alloreactivity as evidenced by a lack of cytotoxicity elicited against the same allogeneic cells that were used for stimulation, whereas CAR19-T_{conv} cells developed high degree of alloreactivity in a dose-dependent manner (FIG. 9b). Meanwhile, co-culturing CAR19-T_{conv} cells with allogeneic CD8+ T cells previously primed with CAR19-T_{conv} cells as shown in FIG. 9c, induced a significant degree of killing in a dose-dependent manner (FIG. 9d), suggesting that allogeneic CAR19-T_{conv} cells can elicit allogeneic response of recipient's immune system and be rejected. In contrast, the number of CAR19-DNTs killed by allogeneic CD8+ T cells stimulated with CAR19-DNT cells were significantly less (FIG. 9c-d), again, supporting that CAR19-DNT cells are less alloreactive and may avoid HvG rejection. To understand how CAR19-DNT cells are avoiding alloreactivity, in vitro assay was conducted where DNT cells were co-cultured with allogeneic CD8+ T cells in the presence of allo-Ags (FIG. 9e). Surprisingly, the presence of DNT cells inhibited the onset of alloreactivity of CD8+ T cells, while those stimulated in the absence of DNTs induced potent alloreactivity (FIG. 9f), suggesting that CAR19-DNT cells are actively suppressing alloreactivity to protect themselves from allo-rejection.

To further determine the safety of allogeneic CAR19-DNTs, naïve NSG mice were untreated or infused with CAR19-DNT cells or CAR19-T_{conv} cells. It was observed that CAR19-T_{conv} cell-treated mice developed signs of GvHD as mice started to lose body weight and showed other signs of sickness, such as hunched back and reduced mobility (FIGS. 10a and 10b). A reduced survival of the mice treated with CAR19-T_{conv} cells was also observed, in contrast to CAR19-DNT group showing no cases of mortality (FIG. 10c). Severe tissue damage was also observed in liver histology of CAR19-T_{conv} cell treated group, but not in untreated and CAR19-DNT cell treated mice (FIG. 10d).

Cytokine release syndrome is a common CAR-T cell associated-toxicities seen in patients (Giavridis et al., 2018), largely mediated by IL-1β and IL-6 produced by monocytes activated by CAR-T cells. To evaluate the impact of CAR-DNT cells on IL-1β and IL-6 production by monocytes relative to CAR-T_{conv} cells, NALM-6 cells were cultured with NT or CAR-transduced -DNT cells or CAR-T_{conv} cells. Subsequently, cytokines produced by the T cells were used to stimulate monocytic cell lines, THP-1 or mTHP-1 for 3-4 day, and the levels of CRS-associated cytokines, IL-1β and IL-6 were measured. A significant increase IL-1β and IL-6 production by monocytic cell lines was observed when stimulated using supernatants produced by CAR19-DNT cells compared to NT-DNTs. However, significantly higher levels of IL-1β and IL-6 obtained when in the presence of cytokines produced by CAR19-T_{conv} cells than that of CAR19-DNT cells (FIGS. 11a and 11b, respectively). Collectively, the results suggest that genetically modified DNT cells including but not limited to CAR19-DNT cells as well as CAR-DNTs that target other antigens such as CD4, CD8 are less likely to cause severe CRS than CAR-T_{conv} cells.

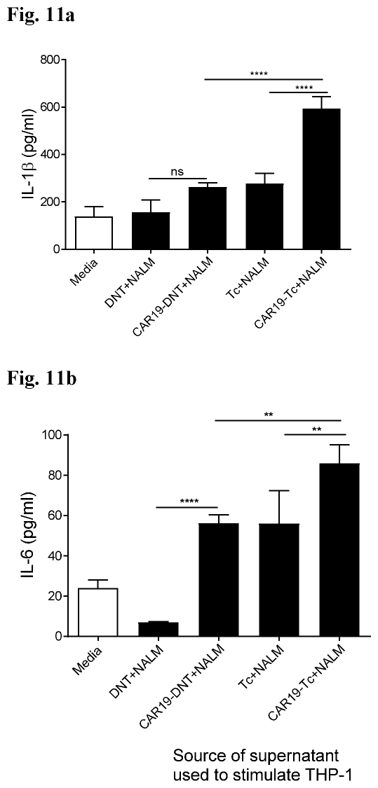

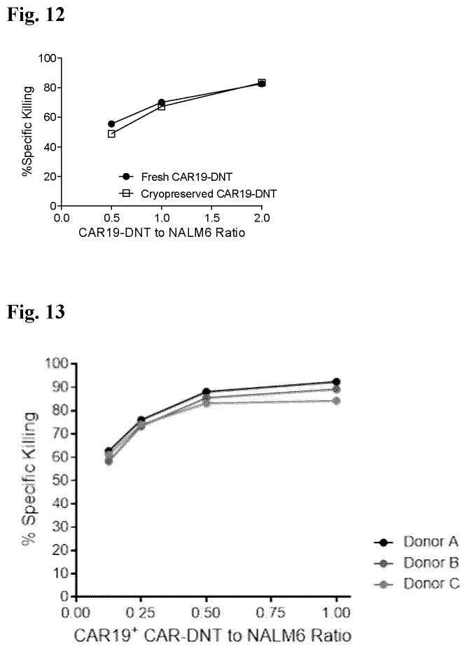

To determine if CAR19-DNT cells retained their off-the-shelf property after cryopreservation, the anti-leukemic activity of cryopreserved CAR19-DNT cells and resistance of CAR19-DNT cells to alloreactivity of T_{conv} cells were determined. CAR19-DNT cells cryopreserved for more than 60 days demonstrate a similar degree of anti-leukemic activity compared to that of fresh CAR19-DNT cells against NALM-6 (FIG. 12).

Previously, it was demonstrated that NT-DNT cells mediate their anti-leukemic activity in a donor-independent manner, fulfilling one of the requirements of an off-the-shelf T cell therapy. To determine whether CAR-DNT cells function in a similar manner, CAR19-DNT cells manufactured using DNT cells obtained from three different donors were used as effector cells against NALM-6 during in vitro cytotoxicity assays. A comparable dose-dependent killing of NALM-6 was observed by all three donors (FIG. 13), supporting that CAR19-DNT cells function in a donor-independent manner, retaining its off-the-shelf potential.

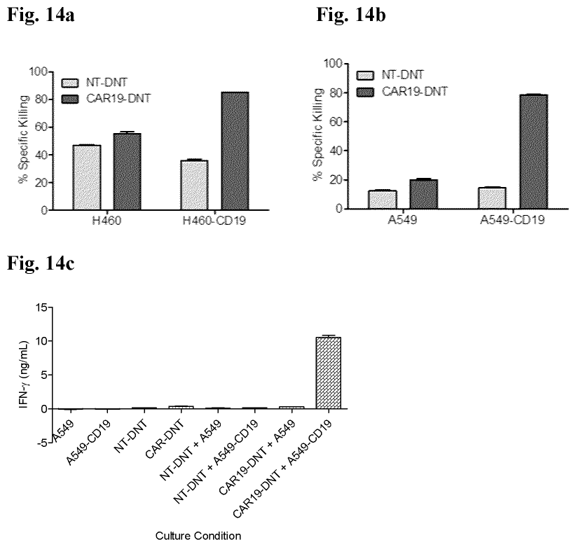

To determine whether CAR-DNT technology would be applicable to other forms of malignancies including solid tumors, the efficacy of CAR19-DNTs to target lung cancer cell lines, A549 and H460, transduced for CD19 expression was tested. Similar to that of B-ALL, CAR19-DNTs induced superior cytotoxic activity against CD19+ A549 and CD19+ H460 than that of NT-DNTs, while CAR19-DNTs and NT-DNTs induced similar degree of cytotoxicity against wild type A549 (FIGS. 14a) and H460 (FIG. 14b). Further, an increase in IFNγ levels in the supernatants was only observed when CAR19-DNT cells were co-cultured with CD19-transduced A549 (FIG. 14c). Collectively, the results demonstrate that DNTs genetically modified with a receptor that recognizes an antigen expressed on solid tumor can significantly enhance their anti-cancer activity.

To further evaluate whether genetically modified DNT cells can effectively target solid cancers in a xenograft model, NSG mice were subcutaneously injected with CD19-transduced A549 cells. Subsequently, mice were untreated or treated with NT- or CAR19-DNT cells. Significantly delayed tumor growth was observed in mice treated with NT-DNT cells relative to the untreated controls, demonstrating incomplete but effective endogenous anti-tumor activity of NT-DNT cells (FIG. 15a). Tumor size from CAR19-DNT cell treated mice was largely unchanged and showed significantly lower fold-change in tumor volume than untreated and NT-DNT cell treated group. Similarly, reduced tumor weights were observed at the end of study for both NT-DNT and CAR19-DNT cells treated mice compared to untreated, with a greater reduction seen with CAR19-DNT cell treatment (FIG. 15b)

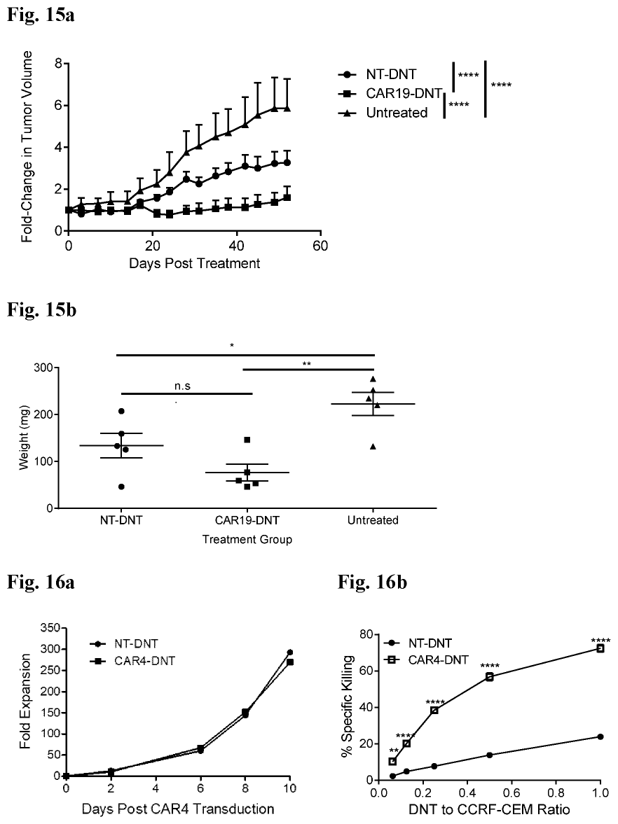

Production of anti-CD4 CAR using conventional T cells to treat T cell leukemia and lymphomas has been difficult due to fratricide of anti-CD4 CAR T cells during production. Additionally, T cell cancers present challenge for making autologous CART product, as the patients' cancerous T cells can contaminate autologous CART products. To determine if allogeneic DNTs can be genetically modified to target T cell cancers such as T-ALL, PTCL and CTCL etc., we took advantage of lack of CD4 expression by DNT cells and developed and transduced allogeneic DNTs with an anti-CD4 CAR (CAR4). We found no signs of fratricide as CAR4-DNTs expanded as good as non-transduced DNTs (FIG. 16a). As expected, CAR4-DNTs more effectively targeted CD4+ T-cell leukemia cell line, CCRF-CEM, than that of NT-DNTs (FIG. 16b). Since CAR4-DNT can be generated from allogeneic healthy donors without contamination of cancerous T cells or fratricide, allogeneic CAR4-DNTs are uniquely well positioned for treating CD4+ T cell cancers. Since DNT cells also do not express CD8, it is expected that anti-CD8 CAR (CAR8) can be transduced to DNTs and used for treating CD8 T cell malignancies.

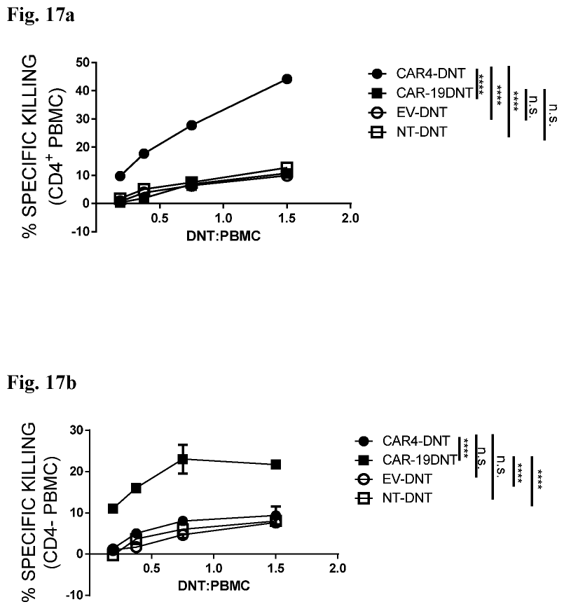

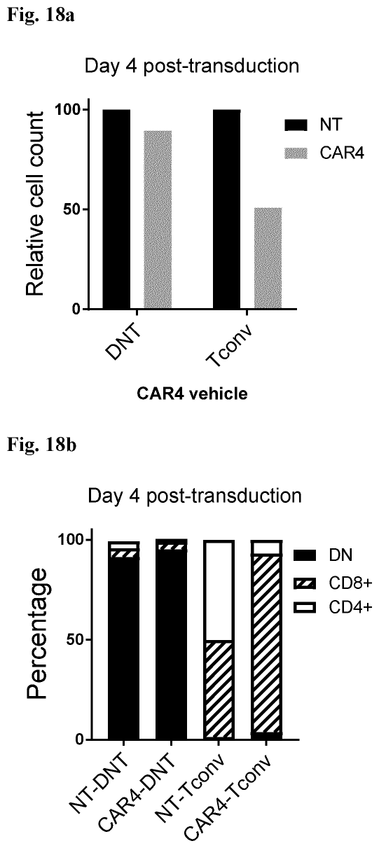

To further evaluate the antigen specificity of CAR4-DNT cells, healthy-donor derived PBMCs were co-cultured with NT or empty-viral vector (EV), CAR19, or CAR4-transduced DNT cells at increasing DNT to PBMC ratio. A significantly improved killing of CAR4-DNT cells against CD4⁺ cells within PBMC was observed, while NT-, EV-, and CAR19-DNT showed minimal toxicity against CD4⁺ PBMCs (FIG. 17a). In contrast, minimal toxicity of CD4-, EV-, NT-DNT cells against CD4⁻ PBMCs was observed, while CD19-DNT cells showed improved toxicity, possibly due to targeting of CD19+ PBMCs. Collectively, these results demonstrate the antigen specificity of CAR4-DNT cells and flexibility of targeting various antigens using different CAR-technology on DNT cell platform. As mentioned above, fratricide is a major hurdle in translation of CAR-T cells for treatment of T cell malignancies. To assess the degree of fratricide during CAR4-T cell manufacturing, the number and composition of NT- or CAR4-transduced DNT or T_{conv} cells was compared. A significant reduction in cell number when T_{conv} cells were transduced with CAR4 was observed (FIG. 18a). Further, significant reductions in the frequency of CD4+ T cells were observed in CAR4-T_{conv} cells compared to NT-T_{conv} cells (FIG. 18b). In contrast, the relative number and composition of CAR4-DNT compared to NT-DNTs were comparable. These results demonstrate that CAR4 can be effectively transduced on DNTs in the absence of fratricide. Populations of CAR4 and CAR8 can therefore be used or administered at much lower doses than NT-DNT and CAR4-T_{conv} and CAR8-T_{conv} for the treatment of cancers.

Collectively, these results support the broad applicability of DNTs as a platform that can 1) be used as an off-the-shelf therapy; 2) adopt different antigen targeting technologies such as CAR-technologies; and 3) be used to treat other liquid and solid cancer types.

### REFERENCES

“FDA Approves Second CAR T-cell Therapy.” (2018). Cancer Discov 8 (1): 5-6.

Alatrash, G. and J. J. Molldrem (2010). “Immunotherapy of AML.” Cancer Treat Res 145: 237-255.

Dwarshuis, N. J., K. Parratt, A. Santiago-Miranda and K. Roy (2017). “Cells as advanced therapeutics: State-of-the-art, challenges, and opportunities in large scale biomanufacturing of high-quality cells for adoptive immunotherapies.” Adv Drug Deliv Rev 114: 222-239.

Giavridis, T., van der Stegen, S. J. C., Eyquem, J. et al. CAR T cell-induced cytokine release syndrome is mediated by macrophages and abated by IL-1 blockade. Nat Med 24,731-738 (2018).

Harris, D. T. and D. M. Kranz (2016). “Adoptive T Cell Therapies: A Comparison of T Cell Receptors and Chimeric Antigen Receptors.” Trends Pharmacol Sci 37(3): 220-230.

He, K. M., Y. Ma, S. Wang, W. P. Min, R. Zhong, A. Jevnikar and Z. X. Zhang (2007). “Donor double-negative Treg promote allogeneic mixed chimerism and tolerance.” Eur J Immunol 37 (12): 3455-3466.

June, C. H., S. R. Riddell and T. N. Schumacher (2015). “Adoptive cellular therapy: a race to the finish line.” Sci Transl Med 7 (280): 280ps287.

Kalos, M. and C. H. June (2013). “Adoptive T cell transfer for cancer immunotherapy in the era of synthetic biology.” Immunity 39 (1): 49-60.

Lee, J., M. D. Minden, W. C. Chen, E. Streck, B. Chen, H. Kang, A. Arruda, D. Ly, S. D. Der, S. Kang, P. Achita, C. D'Souza, Y. Li, R. W. Childs, J. E. Dick and L. Zhang (2017). “Allogeneic Human Double Negative T Cells as a Novel Immunotherapy for Acute Myeloid Leukemia and Its Underlying Mechanisms.” Clin Cancer Res.

Lee, J. B., H. Kang, L. Fang, C. D'Souza, O. Adeyi and L. Zhang (2019). “Developing Allogeneic Double-Negative T Cells as a Novel Off-the-Shelf Adoptive Cellular Therapy for Cancer.” Clin Cancer Res.

Maude, S. L., T. W. Laetsch, J. Buechner, S. Rives, M. Boyer, H. Bittencourt, P. Bader, M. R. Verneris, H. E. Stefanski, G. D. Myers, M. Qayed, B. De Moerloose, H. Hiramatsu, K. Schlis, K. L. Davis, P. L. Martin, E. R. Nemecek, G. A. Yanik, C. Peters, A. Baruchel, N. Boissel, F. Mechinaud, A. Balduzzi, J. Krueger, C. H. June, B. L. Levine, P. Wood, T. Taran, M. Leung, K. T. Mueller, Y. Zhang, K. Sen, D. Lebwohl, M. A. Pulsipher and S. A. Grupp (2018). “Tisagenlecleucel in Children and Young Adults with B-Cell Lymphoblastic Leukemia.” N Engl J Med 378 (5): 439-448.

Maus, M. V., A. K. Thomas, D. G. Leonard, D. Allman, K. Addya, K. Schlienger, J. L. Riley and C. H. June (2002). “Ex vivo expansion of polyclonal and antigen-specific cytotoxic T lymphocytes by artificial APCs expressing ligands for the T-cell receptor, CD28 and 4-1BB.” Nat Biotechnol 20 (2): 143-148.

McIver, Z., B. Serio, A. Dunbar, C. L. O'Keefe, J. Powers, M. Wlodarski, T. Jin, R. Sobecks, B. Bolwell and J. P. Maciejewski (2008). “Double-negative regulatory T cells induce allotolerance when expanded after allogeneic haematopoietic stem cell transplantation.” Br J Haematol 141 (2): 170-178.

Merims, S., X. Li, B. Joe, P. Dokouhaki, M. Han, R. W. Childs, Z. Y. Wang, V. Gupta, M. D. Minden and L. Zhang (2011). “Anti-leukemia effect of ex vivo expanded DNT cells from AML patients: a potential novel autologous T-cell adoptive immunotherapy.” Leukemia 25 (9): 1415-1422.

Neelapu, S. S., F. L. Locke, N. L. Bartlett, L. J. Lekakis, D. B. Miklos, C. A. Jacobson, I. Braunschweig, O. O. Oluwole, T. Siddiqi, Y. Lin, J. M. Timmerman, P. J. Stiff, J. W. Friedberg, I. W. Flinn, A. Goy, B. T. Hill, M. R. Smith, A. Deol, U. Farooq, P. McSweeney, J. Munoz, I. Avivi, J. E. Castro, J. R. Westin, J. C. Chavez, A. Ghobadi, K. V. Komanduri, R. Levy, E. D. Jacobsen, T. E. Witzig, P. Reagan, A. Bot, J. Rossi, L. Navale, Y. Jiang, J. Aycock, M. Elias, D. Chang, J. Wiezorek and W. Y. Go (2017). “Axicabtagene Ciloleucel CAR T-Cell Therapy in Refractory Large B-Cell Lymphoma.” N Engl J Med 377 (26): 2531-2544.

Park, J. H., I. Riviere, M. Gonen, X. Wang, B. Senechal, K. J. Curran, C. Sauter, Y. Wang, B. Santomasso, E. Mead, M. Roshal, P. Maslak, M. Davila, R. J. Brentjens and M. Sadelain (2018). “Long-Term Follow-up of CD19 CAR Therapy in Acute Lymphoblastic Leukemia.” N Engl J Med 378 (5): 449-459.

Ren, J., X. Liu, C. Fang, S. Jiang, C. H. June and Y. Zhao (2017). “Multiplex Genome Editing to Generate Universal CAR T Cells Resistant to PD1 Inhibition.” Clin Cancer Res 23 (9): 2255-2266.

Ruella, M., D. M. Barrett, S. S. Kenderian, O. Shestova, T. J. Hofmann, J. Perazzelli, M. Klichinsky, V. Aikawa, F. Nazimuddin, M. Kozlowski, J. Scholler, S. F. Lacey, J. J. Melenhorst, J. J. Morrissette, D. A. Christian, C. A. Hunter, M. Kalos, D. L. Porter, C. H. June, S. A. Grupp and S. Gill (2016). “Dual CD19 and CD123 targeting prevents antigen-loss relapses after CD19-directed immunotherapies.” J Clin Invest 126 (10): 3814-3826.

Ruella, M. and S. S. Kenderian (2017). “Next-Generation Chimeric Antigen Receptor T-Cell Therapy: Going off the Shelf.” BioDrugs 31 (6): 473-481.

Schuster, S. J., J. Svoboda, E. A. Chong, S. D. Nasta, A. R. Mato, O. Anak, J. L. Brogdon, I. Pruteanu-Malinici, V. Bhoj, D. Landsburg, M. Wasik, B. L. Levine, S. F. Lacey, J. J. Melenhorst, D. L. Porter and C. H. June (2017). “Chimeric Antigen Receptor T Cells in Refractory B-Cell Lymphomas.” N Engl J Med 377 (26): 2545-2554.

Shlomchik, W. D. (2007). “Graft-versus-host disease.” Nat Rev Immunol 7 (5): 340-352.

Sterner R M, Sakemura R, Cox M J, Yang N, Khadka R H, Forsman C L, et al. GM-CSF inhibition reduces cytokine release syndrome and neuroinflammation but enhances CAR-T cell function in xenografts. Blood 2019;133 (7):697-709

Tran, E., P. F. Robbins, Y. C. Lu, T. D. Prickett, J. J. Gartner, L. Jia, A. Pasetto, Z. Zheng, S. Ray, E. M. Groh, I. R. Kriley and S. A. Rosenberg (2016). “T-Cell Transfer Therapy Targeting Mutant KRAS in Cancer.” N Engl J Med 375(23): 2255-2262.

Young, K. J., B. DuTemple, M. J. Phillips and L. Zhang (2003). “Inhibition of Graft-Versus-Host Disease by Double-Negative Regulatory T Cells.” The Journal of Immunology 171 (1): 134-141.

Zeng, J., Tang, S. Y., Toh, L. L. & Wang, S. Generation of ‘Off-the-Shelf’ Natural Killer Cells from Peripheral Blood Cell-Derived Induced Pluripotent Stem Cells. Stem Cell Rep. 9, 1796-1812 (2017).

Zhang, Z. X., L. Yang, K. J. Young, B. DuTemple and L. Zhang (2000). “Identification of a previously unknown antigen-specific regulatory T cell and its mechanism of suppression.” Nat Med 6 (7): 782-789.

Zhao, J., Lin, Q., Song, Y. et al. Universal CARs, universal T cells, and universal CAR T cells. J Hematol Oncol 11, 132 (2018).

1. A method of treating cancer in a subject in need thereof, the method comprising administering to the subject an effective amount of a population of double negative T (DNT) cells that have been genetically modified to bind to one or more target antigens.

2. Use of an effective amount of a population of double negative T (DNT) cells that have been genetically modified to bind to one or more target antigens for treating cancer in a subject in need thereof.

3. The method of claim 1 or the use of claim 2, wherein the DNT cell is genetically modified to express a nucleic acid molecule encoding a chimeric antigen receptor (CAR) that binds to the target antigen.

4. The method or use of any one of claims 1 to 3, wherein the population of genetically modified DNT cells comprises or consists of cells that are CD4−, CD8−, CD3+, γδ-TCR+ and/or αβ-TcR+.

5. The method or use of any one of claims 1 to 4, wherein the population of genetically modified DNT cells comprises or consists of autologous cells.

6. The method or use of any one of claims 1 to 5, wherein the population of genetically modified DNT cells comprises or consists of allogenic cells, optionally from one or more healthy donors.

7. The method or use of any one of claims 1 to 6, wherein the population of genetically modified DNT cells does not induce graft-versus-host disease (GvHD) in the subject or induces less GvHD in the subject relative to conventional T cells or genetically modified conventional T cells.

8. The method or use of any one of claims 1 to 7, wherein the population of genetically modified DNT cells avoids or suppresses host-versus-graft (HvG) rejection in the subject, optionally wherein the population of DNT cells avoids or suppresses HvG rejection in the subject relative to conventional T cells or genetically modified conventional T_{conv} cells.

9. The method or use of claim 8, wherein the population of genetically modified DNT cells persists in the subject for longer than a control population of CD4+ CD8+ CAR T cells, optionally for longer than 2 weeks, 3 weeks, or 4 weeks and/or the population of allogeneic genetically modified DNT cells avoid HvG rejection without the need for additional immunosuppressive therapy.

10. The method or use of any one of claims 1 to 9, wherein the subject does not receive immunosuppressive therapy following administration of the population of genetically modified DNT cells.

11. The method or use of claim 9, wherein the subject does not receive immunosuppressive therapy within 60, 30, 21 or 14 days following administration of the population of genetically modified DNT cells.

12. The method or use of any one of claims 1 to 10, wherein the subject receives lymphodepletion chemotherapy preconditioning prior to administration of the population of genetically modified DNT cells, optionally wherein the lymphodepletion chemotherapy comprises fludarabine and/or cyclophosphamide.

13. The method or use of any one of claims 1 to 11, wherein the population of genetically modified DNT cells comprises DNT cells transduced with a vector, plasmid or mRNA comprising a nucleic acid sequence encoding for one or more chimeric antigen receptors.

14. The method or use of any one of claims 1 to 12, wherein the genetically modified DNT cells are CAR-DNTs and the CAR comprises an extracellular binding domain, a hinge region, a transmembrane domain and/or an intracellular signaling domain.

15. The method or use of any one of claims 1 to 14, wherein the genetically modified DNT cells are CAR-DNTs and the CAR comprises an extracellular antigen-binding domain that binds to a target antigen expressed on a cancer cell in the subject.

16. The method or use of any one of claims 1 to 15, wherein the target antigen is selected from CD4, CD8, CD33, CD19, CD20, CD123 and/or LeY, Mesothelin, EGFR, ROR1, EpCam, MUC1, HER1/2, MET/HGF, neoantigens (driver, non-driver), MAGE family, and NY-ESO-1.

17. The method or use of claim 16, wherein the target antigen is CD4.

18. The method or use of claim 16, wherein the target antigen is CD19.

19. The method or use of any one of claims 1 to 18, wherein the population of genetically modified DNTs have been cryopreserved.

20. The method or use of any one of claims 1 to 19, wherein the cancer is a hematological malignancy, optionally leukemia or lymphoma.

21. The method or use of claim 20, where the cancer is Non-Hodgkin's lymphoma, acute lymphoblastic leukemia, acute myeloid leukemia, or chronic lymphocytic leukemia.

22. The method or use of claim 20, wherein the cancer is acute lymphoblastic leukemia.

23. The method or use of any one of claims 1 to 19, wherein the cancer in the subject comprises one or more solid tumors.

24. The method or use of claim 23, wherein the cancer is lung cancer.

25. The method or use of any one of claims 1 to 24, wherein the cancer is relapsed cancer negative for the target antigen, or where the subject previously received treatment with a population of CAR-T_{conv} cells, optionally treatment with a population of CAR19- or CAR20-T_{conv} cells.

26. The method or use of any one of claims 1 to 25, wherein the cancer exhibits a heterogeneous expression of the target antigens.

27. The method or use of claim 25, wherein the cancer is relapsed acute lymphoblastic leukemia, optionally relapsed B-cell acute lymphoblastic leukemia or relapsed CD19− B-cell acute lymphoblastic leukemia.

28. The method or use of any one of claims 1 to 27, wherein the genetically modified DNT cells are CAR-DNTs and the CAR-DNTs exhibit CAR-targeted and CAR-independent killing of cancer cells in the subject.

29. The method or use of any one of claims 1 to 27, wherein the genetically modified DNTs are not genetically modified to reduce or eliminate expression of one or more genes selected from genes encoding for HLA, T cell receptor CD7, and CD52.

30. The method or use of any one of claims 1 to 29, wherein cytokines produced by the population of genetically modified DNTs stimulate a lower level of production of IL-1β and/or IL-6 by monocytes relative to cytokines produced by conventional T cells (T_{conv}), optionally CAR-T_{conv} cells.

31. The method or use of any one of claims 1 to 30, wherein the population of genetically modified DNT cells does not induce severe cytokine release syndrome (CRS) in the subject or induces less CRS in the subject relative conventional T cells (T_{conv}), optionally CAR-T_{conv} cells.

32. A method of treating CD4+ cancer in a subject in need thereof, the method comprising administering to the subject an effective amount of a population of DNT cells that have been genetically modified to bind to a CD4 target antigen.

33. Use of a population of DNT cells that have been genetically modified to bind to a CD4 target antigen for treating CD4+ cancer in a subject in need thereof.

34. The method of claim 32 or the use of claim 33, wherein the genetically modified DNT cells are CD4-targeting chimeric antigen receptor (CAR)-DNT cells (CAR4-DNT cells).

35. The method or use of any one of claims 32 to 34, wherein the CD4+ cancer is T cell acute lymphoblastic leukemia (T-ALL), peripheral T cell lymphoma (PTCL), or cutaneous T cell lymphoma (CTCL).

36. The method or use of any one of claims 32 to 35, wherein the population of genetically modified DNT cells comprises or consists of cells that are CD4−, CD8−, CD3+, γδ-TCR+ and/or αβ-TcR+.

37. The method or use of any one of claims 32 to 36, wherein the population of genetically modified DNT cells do not induce fratricide or induce less fratricide relative to a population of CAR4 transduced conventional T cells.

38. The method or use of any one of claims 32 to 37, wherein the population of genetically modified DNT cells comprises or consists of allogenic cells, optionally from one or more healthy donors.

39. The method or use of any one of claims 31 to 35, wherein the population of genetically modified DNT cells does not induce graft-versus-host disease (GvHD) in the subject or induces less GvHD in the subject relative to conventional T cells or CAR-T_{conv} cells

40. The method or use of any one of claims 32 to 39, wherein the population of genetically modified DNT cells avoids or suppresses host-versus-graft (HvG) rejection in the subject optionally wherein the population of genetically modified DNT cells avoid or suppresses HvG rejection in the subject relative to conventional T cells or CAR-T_{conv} cells.

41. The method or use of any one of claims 32 to 40, wherein the population of genetically modified DNT cells persists in the subject for longer than a control population of CAR4 CD4+ CD8+ T cells (CAR4-T_{conv} cells), optionally for longer than 2 weeks, 3 weeks, or 4 weeks and/or wherein the population of allogeneic genetically modified DNT cells avoid or suppress HvG rejection without the need of additional immunosuppressive therapies.

42. The method or use of any one of claims 32 to 41, wherein the subject does not receive immunosuppressive therapy following administration of the population of genetically modified DNT cells.

43. The method or use of claim 42, wherein the subject does not receive immunosuppressive therapy within 60, 30, 21 or 14 days following administration of the population of genetically modified DNT cells.

44. The method or use of any one of claims 32 to 43, wherein the cancer is relapsing cancer and the subject previously received treatment with a population of CAR-T_{conv} cells, or wherein the cancer is relapsed cancer negative for the CD4 target antigen.

45. The method or use of any one of claims 32 to 44, wherein the cancer exhibits a heterogeneous expression of the CD4 target antigen.

46. The method or use of any one of claims 32 to 45, wherein the genetically modified DNTs are not genetically modified to reduce or eliminate expression of one or more genes selected from genes encoding for HLA, endogenous T cell receptor, CD7, and CD52.

47. The method or use of any one of claims 32 to 46, wherein cytokines produced by the population of genetically modified DNTs stimulates a lower level of production of IL-1β and/or IL-6 by monocytes relative to cytokines produced by CARO-T_{conv} cells.

48. The method or use of any one of claims 32 to 47, wherein the population of genetically modified DNT cells does not induce cytokine release syndrome (CRS) in the subject or induces less CRS in the subject relative to CAR4-T_{conv} cells.

49. A double negative T (DNT) cell that has been genetically modified to bind to a target antigen.

50. The genetically modified DNT cell of claim 49, wherein the DNT cell is genetically modified to express a nucleic acid sequence encoding a chimeric antigen receptor (CAR) that binds to the target antigen.

51. The genetically modified DNT cell of claim 49 or 50, wherein the DNT cell is CD4−, CD8−, CD3+, γδ-TCR+ and/or αβ-TcR+.

52. The genetically modified DNT cell of any one of claims 49 to 51, wherein allogenic populations of the genetically modified DNT cells do not induce graft-versus-host disease (GvHD) in a subject or induces less GvHD in the subject relative to conventional T cells or conventional CAR-T cells (CAR-T_{conv} cells).

53. The genetically modified DNT cell of any one of claims 49 to 52, wherein allogenic populations of the genetically modified DNT cells avoid or suppress host-versus-graft rejection in a subject, optionally wherein the population of CAR-DNT cells suppresses HvG rejection in the subject relative to CAR-T_{conv} cells.

54. The genetically modified DNT cell of any one of claims 49 to 53, wherein the DNT cell is transduced with a vector, plasmid or mRNA, optionally comprising the nucleic acid sequence encoding the CAR.

55. The genetically modified DNT cell of any one of claims 49 to 54, wherein the CAR comprises an extracellular binding domain, a hinge region, a transmembrane domain and/or an intracellular signaling domain.

56. The genetically modified DNT cell of any one of claims 49 to 55, wherein the CAR comprises an extracellular antigen binding domain that binds to a target antigen expressed on a cancer cell.

57. The genetically modified DNT cell of claim 56, wherein the DNT cell is genetically modified to bind to a target antigen is selected from CD4, CD8, CD33, CD19, CD20, CD123, LeY, Mesothelin, EGFR, ROR1, EpCam, MUC1, HER1/2, MET/HGF, neoantigens (driver, non-driver), MAGE family and NY-ESO-1.

58. The genetically modified DNT cell of claim 57, wherein the target antigen is CD4.

59. The genetically modified DNT cell of claim 57, wherein the target antigen is CD19.

60. The genetically modified DNT cell of any one of claims 49 to 59, wherein the CAR-DNT cell has been cryopreserved.

61. The genetically modified DNT cell of any one of claims 49 to 60, wherein the CAR-DNT is not genetically modified to reduce or eliminate expression of one or more genes selected from genes encoding for HLA, endogenous T cell receptor, CD7, or CD52.

62. The genetically modified DNT cell of any one of claims 49 to 61, wherein cytokines produced by a population of the genetically modified DNT cells stimulate a lower level of production of IL-1β and/or IL-6 by monocytes relative to cytokines produced by CAR-T_{conv} cells.

63. The genetically modified DNT cell of any one of claims 49 to 62, wherein a population of the genetically modified DNT cells does not induce cytokine release syndrome (CRS) or induces less CRS relative to CAR-T_{conv} cells.

64. A composition comprising a population of genetically modified DNT cells of any one of claims 49 to 63 and a pharmaceutically acceptable carrier.

65. Use of the composition of claim 64 for the treatment of cancer in a subject in need thereof.
